(19) United States
(12) Patent Application Publication
(10) Pub. No.: US 2022/0289820 A1
(43) Pub. Date: Sep. 15, 2022

(54) CHIMERIC CYTOKINE RECEPTOR

(71) Applicant: AUTOLUS LIMITED, London (GB)

(72) Inventors: Martin Pulé, London (GB); Shaun Cordoba, London (GB); Simon Thomas, London (GB); Shimobi Onouha, London (GB); Matteo Righi, London (GB)

(21) Appl. No.: 17/628,509

(22) PCT Filed: Aug. 4, 2020
(86) PCT No.: PCT/GB2020/051865

§ 371 Date: Jan. 19, 2022

(30) Foreign Application Priority Data
Aug. 5, 2019 (GB) 1911187.1

(51) Int. Cl. C07K 14/735; C07K 14/715; C07K 16/30; A61P 35/00

(52) U.S. Cl. CPC C07K 14/70535; C07K 14/7155; C07K 16/3069; A61P 35/00; C07K 2317/52; C07K 2317/622; C07K 2319/03; C07K 2319/30

(57) ABSTRACT

The present invention provides a chimeric cytokine receptor comprising a cytokine receptor endodomain which comprises a first chain and a second chain, wherein the first and/or second chain of the cytokine-receptor endodomain is/are truncated. The invention also provides cells comprising such a chimeric cytokine receptor, optionally in combination with a chimeric antigen receptor (CAR) and their use in the treatment of diseases such as cancer.

### FIELD OF THE INVENTION

The present invention relates to chimeric cytokine receptors (CCRs). In particular, the present invention relates to CCRs in which one or more chains of the cytokine receptor is truncated.

### BACKGROUND TO THE INVENTION

Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

CAR-Based Approaches to Treat Prostate Cancer

Prostate cancer is the second most common cancer in men worldwide, and the sixth leading cause of cancer-related death. Globally, there are approximately 1,100,000 new cases and 300,000 mortalities every year, comprising 4 percent of all cancer deaths. It is estimated that 1 in every 6 men will be diagnosed with the disease during his lifetime.

Initial treatment for prostate cancer may consist of surgery, radiation, or hormone therapy, or any combination of each. Hormone therapy consists of lowering the levels of testosterone, the male hormone that fuels out-of-control cell growth. Chemotherapy is typically reserved for advanced-stage cancers.

When prostate cancers grow despite the lowering of testosterone levels by hormone therapy, treatment options are limited. Typically, the cancer vaccine sipuleucel-T (Provenge®) a dendritic cell-based therapeutic cancer vaccine designed to induce an immune response targeted against the prostatic acid phosphatase ((PAP) antigen), a radiopharmaceutical agent (such as radium-223 chloride), secondary hormone therapies (such as abiraterone or enzalutamide), and/or chemotherapies (docetaxel and cabazitaxel) are added to the hormonal therapy in sequence. While each of these treatments can delay growth of the cancer for several months and palliate symptoms produced by the disease, the disease ultimately becomes resistant to them.

Preclinically, two antigens associated with prostate cancer have been targeted with CAR T-cell based therapies: prostate-specific membrane antigen (PSMA) and prostate stem cell antigen (PSCA).

Mice treated with PSCA CAR-engineered T cells showed delayed tumour growth (Hillerdal et al (2014) BMC Cancer 14:30; and Abate-Daga et al (2014) 25:1003-1012). Although the cells showed high in vitro cytotoxicity, in vivo, tumour growth was delayed but tumour-bearing mice were not cured.

This may be because, in vivo, CAR T-cells struggle to overcome the hostile microenvironment of a carcinoma. In particular CAR T-cells may fail to engraft and expand within a prostate cancer tumour bed.

CAR T-cell persistence and activity can be enhanced by administration of cytokines, or by the CAR T-cells producing cytokines constitutively. However, these approaches have limitations: systemic administration of cytokines can be toxic; constitutive production of cytokines may lead to uncontrolled proliferation and transformation (Nagarkatti et al (1994) PNAS 91:7638-7642; Hassuneh et al (1997) Blood 89:610-620).

There is therefore a need for alternative CAR T-cell approaches, which facilitate engraftment and expansion of T cells to counteract the effects of the hostile tumour microenvironment.

On-Target Off-Tumour Toxicity

It is relatively rare for the presence of a single antigen effectively to describe a cancer, which can lead to a lack of specificity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been borne out by clinical trials. For example, an approach targeting ERBB2 caused death to a patient with colon cancer metastatic to the lungs and liver. ERBB2 is over-expressed in colon cancer in some patients, but it is also expressed on several normal tissues, including heart and normal vasculature.

There is therefore a need for improved approaches to cancer therapy in which such “on-target off-tumour” toxicity is reduced or eliminated.

WO2017/029512 describes two types of chimeric cytokine receptor (CCR). The first type of CCR grafts the binding specificity of a non-cytokine binding molecule on to the endodomain of a cytokine receptor. In the presence of the ligand for the CCR, a cytokine signal is delivered to the CCR-expressing cell. The second type of CCR comprises a dimerization domain and a cytokine receptor endodomain. Dimerisation may occur spontaneously, in which case the chimeric transmembrane protein will be constitutively active. Alternatively, dimerization may occur only in the presence of a chemical inducer of dimerization (CID) in which case the transmembrane protein only causes cytokine-type signalling in the presence of the CID.

The co-expression of such a CCR with a chimeric antigen receptor (CAR) helps a CAR T-cell to engraft and expand in the hostile tumour microenvironment.

The expansion of CCR transduced cells in vivo will occur if the rate of proliferation is higher than the rate of cell death. Cells will reach homogenization (steady-state) when the rate of proliferation is equal to the cellular death rate. When the rate of death is higher than the rate of proliferation the cells will not persist. In some instances, a super-physiological activation of the CCR may be required to ensure cells persist in vivo. In other instances, a reduced proliferation may be required to match cellular death rates to maintain cellular homogeny. In other instances, some cells may be hypersensitive to CCR signals and excessive activation of the CCR may alter cellular function or differentiation and a reduced CCR signal may be required.

### DESCRIPTION OF THE FIGURES

FIG. 1: Schematic diagram summarising the structure of various cytokine receptors, the cell types which produce the cytokines and the cell types which express the cytokine receptors.

FIG. 2: Schematic diagram showing proposed chimeric cytokine receptor

(a) Cytokine IL2 and IL7 cytokine receptors signal through a common gamma chain and a cytokine specific alpha/beta chain.

(b) One implementation of a chimeric cytokine receptor is to replace the ectodomain of the cytokine alpha/beta and gamma chain with different scFvs (or any other suitable binder) which recognize different epitopes of PSA.

(c) An alternative approach is to replace the ectodomains of alpha/beta and gamma with the VH/VL of a PSA specific antibody, where both VH and VL are involved in binding so that binding brings them together.

FIG. 3: Aggregation-based cytokine signalling enhancer

Schematic diagram showing a chimeric cytokine receptor and CAR combination system. The cell comprises two chimeric cytokine receptors which bind different epitopes on the same soluble ligand. In the absence of soluble ligand (e.g. PSA) but the presence of the cell-membrane antigen (e.g. PSMA) signalling occurs thought the CAR. In the presence of the soluble ligand, aggregation of the two chimeric cytokine receptors occurs, leading to cytokine-based signal enhancement.

FIG. 4: Theoretical construct map for the chimeric cytokine receptor/CAR combination system illustrated in FIG. 3.

FIG. 5: Schematic diagram illustrating an example of a structure for the chimeric transmembrane protein of the present invention. The chimeric transmembrane protein comprises a dimerization domain and a cytokine receptor endodomain. The embodiment shown has a “Fab” type architecture, as the dimerization domain comprises antibody-type heavy and light chain constant regions. Constant dimerization between these domains brings together the IL2 receptor common γ chain with either the IL-2 receptor β chain or the IL-7 receptor α chain, leading to constitutive cytokine signalling.

FIG. 6: IL-2 signalling by the chimeric transmembrane protein.

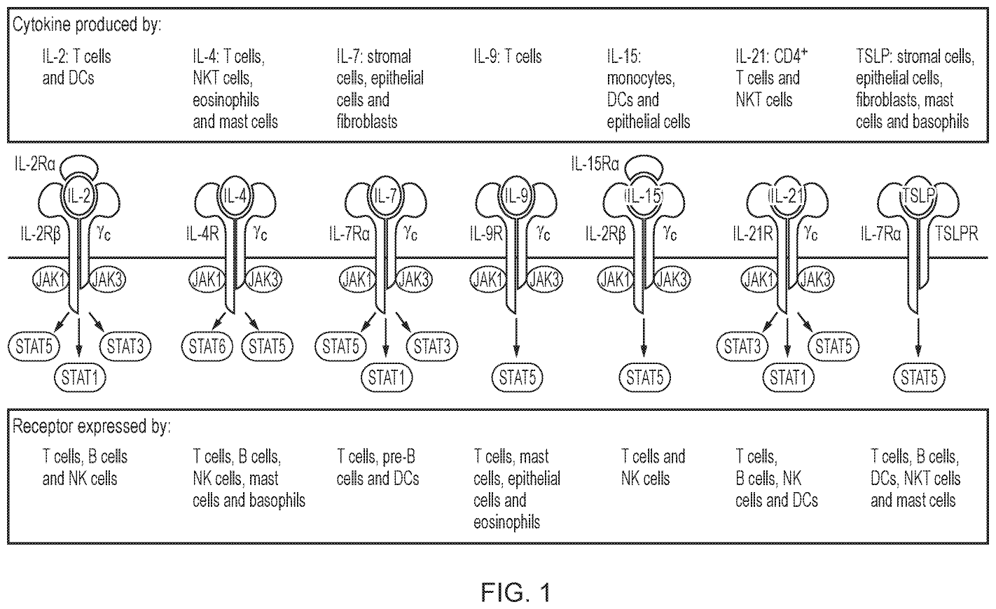

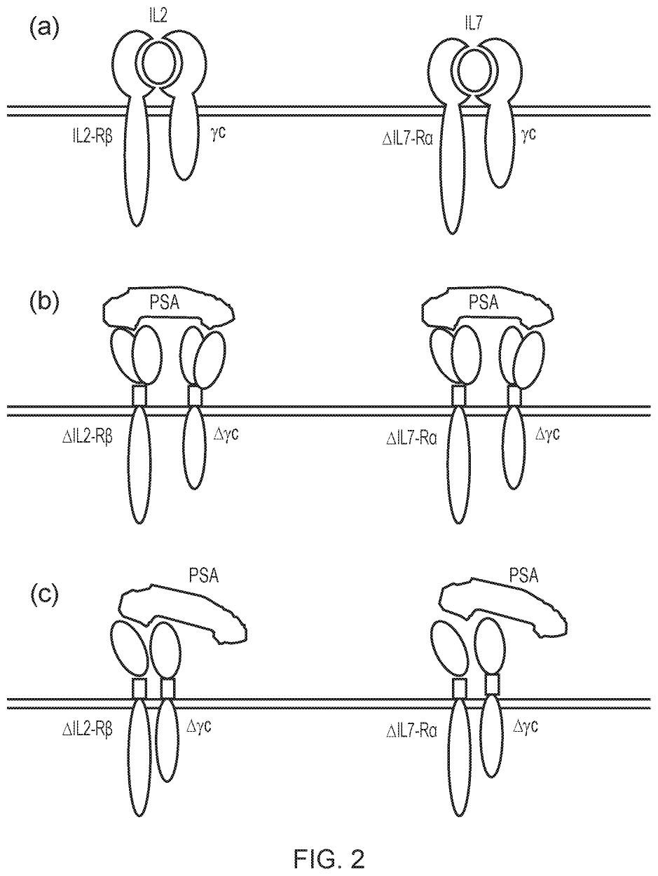

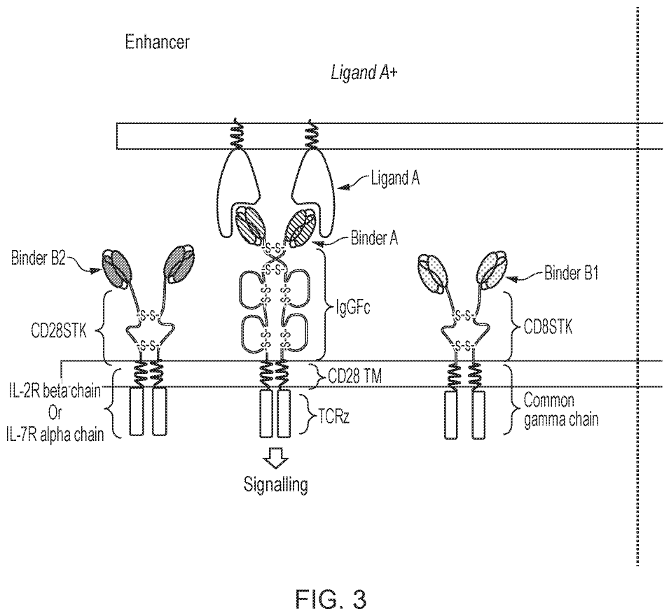

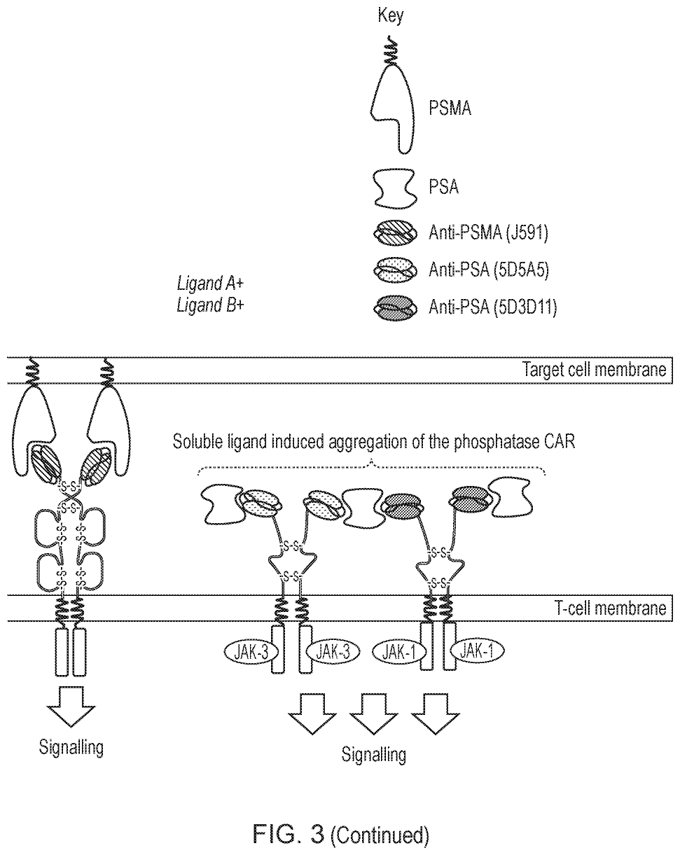

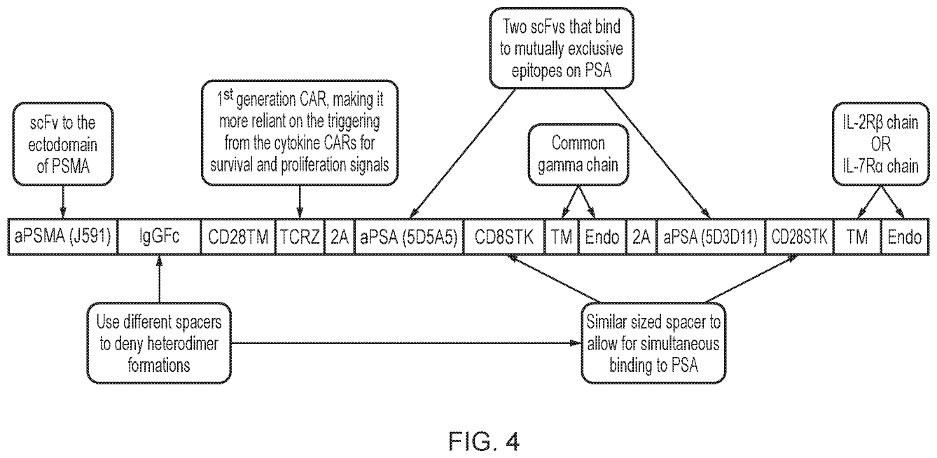

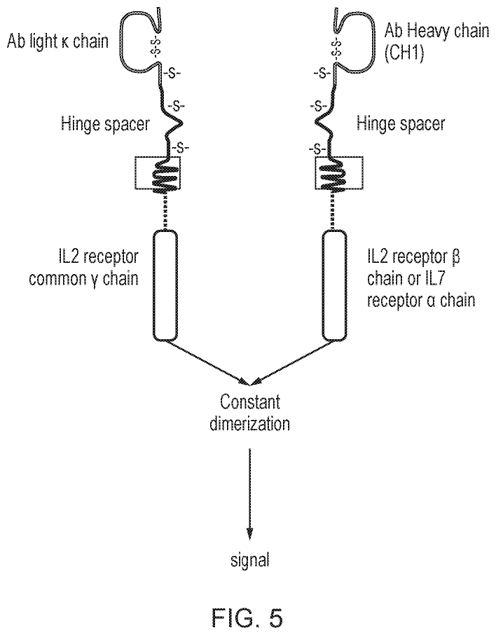

Two chimeric transmembrane proteins having the general structure shown in FIG. 5 were tested for their ability to induce IL-2 signalling. One chimeric transmembrane protein comprised an IL2 receptor endodomain and the other comprised an IL-7 receptor endodomain. IL-2 signalling was tested using the murine cell line CTLL2 which is dependent on IL-2 signalling for growth. As a positive control, CTLL2 cells were cultured with 100 u/mL murine IL2. Cells expressing the chimeric transmembrane protein comprising the IL2 receptor endodomain (Fab_IL2endo) supported CTLL2 cell survival and growth, whereas cells expressing the chimeric transmembrane protein comprising the IL-7 receptor (Fab_IL7endo) did not.

FIG. 7: Schematic diagram illustrating panel of PSA chimeric cytokine receptors A panel of chimeric cytokine receptors (CCRs) targeting PSA was developed using scFvs derived from two antibodies which bind to different PSA epitopes: 5D5A5 and 5D3D11.

Top-left panel: A CCR with an IL-2R endodomain having A5 on the chain with IL2R β chain and D11 on the chain with common γ chain;

Top-right panel: A CCR with an IL7R endodomain having A5 on the chain with IL7R α chain and D11 on the chain with common γ chain;

Bottom-left panel: A CCR with an IL-2R endodomain having D11 on the chain with IL2R β chain and A5 on the chain with common γ chain; and

Bottom-right hand panel: A CCR with an IL-7R endodomain having D11 on the chain with IL7R α chain and A5 on the chain with common γ chain.

A negative control was also created for each CCR, in which the IL2Rγ chain was replaced by a rigid linker.

FIG. 8: IL2 signalling from cells expressing a PSA chimeric cytokine receptor in the presence of PSA-CTLL2 proliferation

CTLL2 cells were transduced with constructs expressing some of the PSA chimeric cytokine receptors illustrated in FIG. 7. Cells were cultured in the presence of absence of IL2 (positive control) and the presence of absence of 5 ng/mL or 5 μg/mL PSA. CTLL2 proliferation was assessed after 3 and 7 days.

The anti-PSA chimeric cytokine receptor with an IL2R endodomain supported CTLL2 cell proliferation in the absence of IL2 and the presence of PSA, but not the receptor having an IL7R endodomain or any of the CCRs comprising a rigid linker in the place of the common γ chain.

FIG. 9: IL2 signalling from cells expressing a PSA chimeric cytokine receptor in the presence of PSA-CTLL2 STAT5 phosphorylation

CTLL2 cells were either left untransduced (WT); or transduced with a vector expressing a CCR against PSA (D11-CD8STK-IL2Rg_A5-Hinge-IL2Rb) or an equivalent construct having a rigid linker in the place of the common γ chain (D11-CD8STK-RL_A5-Hinge-IL2Rb). Cells were incubated with either 500 μM Pervanadate or 500 ng/mL PSA for 1 or 4 hours. Phosphorylation of Y694 of STAT5 was then investigated using phosphoflow.

FIG. 10: Proliferation signal mediated by IL2R beta chain truncations. a) Diagrammatic representation of the different truncations of the IL2R beta chain. Each truncation was paired with the full length IL2R common gamma chain. b) Transduced T cells were cultured for 4 days in absence of exogenous cytokines (starvation assay). The absolute number of viable, transduced cells was assessed by flow cytometry. These values were normalized to the value acquired on day 0 and plotted on the y-axis as a fold change from day 0. Boxes represent the median value of 4 separate donors.

FIG. 11: The general structure of a receptor from the type I cytokine receptor family. In the extracellular cytokine receptor module, four conserved cysteine residues exist and are involved in disulfide bonds. A WSXWS (Tre, Ser, any, Tre, Ser) motif that is essential for receptor processing, ligand binding, and activation of the receptor is also located in the extracellular domain. In the intracellular portion, two short domains termed Box 1 and Box 2 are important for JAK binding. Tyrosine residues are present on the intracellular part which are phosphorylated upon receptor activation.

### SUMMARY OF ASPECTS OF THE INVENTION

The present inventors have found that it is possible to alter the cytokine signal generated by a chimeric cytokine receptor by truncating one or both chains of the cytokine receptor endodomain. Surprisingly, the initial deletion improved cellular proliferation and subsequent longer deletions cause cytokine signalling to be reduced in an analog manner, so it is possible to choose the desired level of cytokine signalling by selecting the appropriate truncation.

Thus, in a first aspect, the present invention provides a chimeric transmembrane protein comprising:

a dimerization domain; and

a truncated endodomain from a cytokine receptor.

The dimerization domain may comprise the dimerization portion of a heavy chain constant domain (CH) or a light chain constant domain (CL).

In a second aspect, the present invention provides a chimeric cytokine receptor comprising a cytokine receptor endodomain which comprises a first chain and a second chain, wherein the first and/or second chain of the cytokine-receptor endodomain is/are truncated.

In a first embodiment of the second aspect of the invention there is provided a chimeric cytokine receptor which comprises two polypeptides:
(i) a first polypeptide which comprises:
   (a) a first dimerisation domain; and
   (b) a first chain of the cytokine receptor endodomain; and
(ii) a second polypeptide which comprises:
   (a) a second dimerization domain, which dimerises with the first dimerization domain; and
   (b) a second chain of the cytokine-receptor endodomain
wherein the first and/or second chain of the cytokine-receptor endodomain is/are truncated.

The first and second dimerization domains may dimerise spontaneously.

Alternatively, the first and second dimerization domains dimerise in the presence of a chemical inducer of dimerization (CID) or the presence of a protein.

The chimeric cytokine receptor may comprise two polypeptides:
(i) a first polypeptide which comprises:
   (a) a heavy chain constant domain (CH)
   (b) a first chain of the cytokine receptor endodomain; and
(ii) a second polypeptide which comprises:
   (a) a light chain constant domain (CL)
   (b) a second chain of the cytokine-receptor endodomain.

There is also provided a provided a chimeric cytokine receptor comprising:
an exodomain which binds to a ligand; and
a cytokine receptor endodomain comprising a first chain and a second chain wherein the first and/or second chain of the cytokine-receptor endodomain is/are truncated.

In a second embodiment of the second aspect of the invention, there is provided a chimeric cytokine receptor which comprises two polypeptides:
(i) a first polypeptide which comprises:
   (a) a first antigen-binding domain which binds a first epitope of the ligand
   (b) a first chain of the cytokine receptor endodomain; and
(ii) a second polypeptide which comprises:
   (a) a second antigen-binding domain which binds a second epitope of the ligand
   (b) a second chain of the cytokine-receptor endodomain.

Each of the first and second antigen-binding domains may be, for example, single-chain variable fragments (scFvs) or single domain binders (dAbs).

In a third embodiment of the second aspect of the invention there is provided a chimeric cytokine receptor which comprises two polypeptides:
(i) a first polypeptide which comprises:
   (a) a heavy chain variable domain (VH)
   (b) a first chain of the cytokine receptor endodomain; and
(ii) a second polypeptide which comprises:
   (a) a light chain variable domain (VL)
   (b) a second chain of the cytokine-receptor endodomain.

Where the chimeric cytokine receptor comprises an exodomain which binds a ligand, the ligand may, for example, be a tumour secreted factor selected from: prostate-specific antigen (PSA), carcinoembryonic antigen (CEA) and vascular endothelial growth factor (VEGF) and CA125.

Alternatively the ligand may be a chemokine selected from: CXCL12, CCL2, CCL4, CCL5 and CCL22.

The first and second chains of the cytokine receptor endodomain may be selected from type I cytokine receptor endodomain α-, β-, and γ-chains. For example, the first and second chains of the cytokine receptor endodomain may be selected from:

(i) IL-2 receptor β-chain endodomain

(ii) IL-7 receptor α-chain endodomain; or

(iii) IL-15 receptor α-chain endodomain; and/or

(iv) common γ-chain receptor endodomain.

In particular, the chimeric cytokine receptor may comprise a truncated IL-2 receptor β-chain endodomain.

In a third aspect, the present invention provides a cell which comprises a chimeric transmembrane protein according to the first aspect of the invention or a chimeric cytokine receptor according to the second aspect of the invention.

The cell may also comprise a chimeric antigen receptor.

In a fourth aspect, the present invention provides a nucleic acid sequence encoding a chimeric transmembrane protein the first aspect of the invention.

In a fifth aspect, there is provided a nucleic acid construct encoding a chimeric cytokine receptor according to the second aspect of the invention.

A nucleic acid construct encoding a chimeric cytokine receptor according to the first embodiment of the second aspect of the invention may comprise a first nucleic acid sequence encoding the first polypeptide; and a second nucleic acid sequence encoding the second polypeptide, the nucleic acid construct having the structure:

Dim1-TM1-endo1-coexpr-Dim2-TM2-endo2

in which

Dim1 is a nucleic acid sequence encoding the first dimerisation domain;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both CCRs

Dim2 is a nucleic acid sequence encoding the second dimerization domain;

TM2 is a nucleic acid sequence encoding the transmembrane domain of the second polypeptide;

endo 2 is a nucleic acid sequence encoding the endodomain of the second polypeptide.

A nucleic acid construct encoding a chimeric cytokine receptor according to the second embodiment of the second aspect of the invention may comprise a first nucleic acid sequence encoding the first polypeptide and a second nucleic acid sequence encoding the second polypeptide, the nucleic acid construct having the structure:

AgB1-spacer1-TM1-endo1-coexpr-AbB2-spacer2-TM2-endo2

in which

AgB1 is a nucleic acid sequence encoding the antigen-binding domain of the first polypeptide;

spacer 1 is a nucleic acid sequence encoding the spacer of the first polypeptide;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both polypeptides

AgB2 is a nucleic acid sequence encoding the antigen-binding domain of the second polypeptide;

spacer 2 is a nucleic acid sequence encoding the spacer of the second polypeptide;

TM2 is a nucleic acid sequence encoding the transmembrane domain of the second polypeptide;

endo 2 is a nucleic acid sequence encoding the endodomain of the second polypeptide.

A nucleic acid construct encoding a chimeric cytokine receptor according to the third embodiment of the second aspect of the invention may comprise a first nucleic acid sequence encoding the first polypeptide and a second nucleic acid sequence encoding the second polypeptide, the nucleic acid construct having the structure:

VH-spacer1-TM1-endo1-coexpr-VL-spacer2-TM2-endo2

in which

VH is a nucleic acid sequence encoding the VH domain of the first polypeptide; spacer 1 is a nucleic acid sequence encoding the spacer of the first polypeptide;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both polypeptides

VL is a nucleic acid sequence encoding the VL domain of the second polypeptide;

spacer 2 is a nucleic acid sequence encoding the spacer of the second polypeptide;

TM2 is a nucleic acid sequence encoding the transmembrane domain of the second polypeptide;

endo 2 is a nucleic acid sequence encoding the endodomain of the second polypeptide.

The nucleic acid construct may also encode a chimeric antigen receptor (CAR).

The “coexpr” sequence may encode a sequence comprising a self-cleaving peptide.

Alternative codons may be used in regions of sequence encoding the same or similar amino acid sequences, in order to avoid homologous recombination.

In a sixth aspect, the present invention provides a vector comprising a nucleic acid sequence according to the fourth aspect of the invention or a nucleic acid construct according to the fifth aspect of the invention.

In a seventh aspect, the present invention provides a kit which comprises:
i) a vector comprising a nucleic acid sequence encoding a first polypeptide of a CCR according to the second aspect of the invention; and
ii) a vector comprising a nucleic acid sequence encoding a second polypeptide of a CCR according to the second aspect of the invention.

The kit may also comprise a vector comprising a nucleic acid sequence encoding a chimeric antigen receptor.

In an eighth aspect, the present invention provides method for making a cell according to the third aspect of the invention, which comprises the step of introducing: a nucleic acid sequence according to the fourth aspect of the invention; a nucleic acid construct according to the fifth aspect of the invention; a vector according to the sixth aspect of the invention; or a kit of vectors according to the seventh aspect of the invention, into a cell ex vivo.

The cell may be from a sample isolated from a subject.

In a ninth aspect, the present invention provides a pharmaceutical composition comprising a plurality of cells according to the third aspect of the invention.

In a tenth aspect, the present invention provides a method for treating and/or preventing a disease, which comprises the step of administering a pharmaceutical composition according to the ninth aspect of the invention to a subject.

The method may comprise the following steps:
(i) isolation of a cell-containing sample from a subject;
(ii) transduction or transfection of the cells with: a nucleic acid sequence according to the fourth aspect of the invention; a nucleic acid construct according to the fifth aspect of the invention; a vector according to the sixth aspect of the invention; or a kit of vectors according to the seventh aspect of the invention; and
(iii) administering the cells from (ii) to the subject.

The sample may be a T-cell containing sample.

The disease may be a cancer.

In an eleventh aspect, the present invention provides a pharmaceutical composition according to the ninth aspect of the invention for use in treating and/or preventing a disease.

In a twelfth aspect, the present invention provides the use of a cell according to the third aspect of the invention in the manufacture of a medicament for treating and/or preventing a disease.

### DETAILED DESCRIPTION

Chimeric Cytokine Receptor (CCR)

Chimeric cytokine receptors are described in WO2017/029512.

A chimeric cytokine receptor (CCR) is a molecule which comprises a cytokine receptor endodomain and either a heterologous ligand-binding exodomain or a dimerization domain, which brings the two chains of the cytokine receptor endodomain together. This latter type of CCR is discussed in more detail below.

In ligand binding-type CCRs, the heterologous exodomain binds a ligand other than the cytokine for which the cytokine receptor from which the endodomain was derived is selective. In this way, it is possible to alter the ligand specificity of a cytokine receptor by grafting on a heterologous binding specificity.

A ligand-binding chimeric cytokine receptor comprises:

(i) a ligand binding exodomain;

(ii) an optional spacer;

(iii) a transmembrane domain; and

(iv) a cytokine-receptor endodomain.

The present invention also provides a chimeric transmembrane protein comprising a dimerization domain; and a cytokine receptor endodomain.

Dimerisation of such a protein can produce a chimeric cytokine receptor. This type of chimeric cytokine receptor comprises:

(i) a dimerising exodomain;

(ii) an optional spacer;

(iii) a transmembrane domain; and

(iv) a cytokine-receptor endodomain.

Dimerisation may occur spontaneously, in which case the chimeric transmembrane protein will be constitutively active. Alternatively, dimerization may occur only in the presence of a chemical inducer of dimerization (CID) in which case the transmembrane protein only causes cytokine-type signalling in the presence of the CID.

Suitable dimerization domains and CIDs are described in WO2015/150771, the contents of which are hereby incorporated by reference.

For example, one dimerization domain may comprise the rapamycin binding domain of FK-binding protein 12 (FKBP12), the other may comprise the FKBP12-Rapamycin Binding (FRB) domain of mTOR; and the CID may be rapamycin or a derivative thereof.

One dimerization domain may comprise the FK506 (Tacrolimus) binding domain of FK-binding protein 12 (FKBP12) and the other dimerization domain may comprise the cyclosporin binding domain of cylcophilin A; and the CID may be an FK506/cyclosporin fusion or a derivative thereof.

One dimerization domain may comprise an oestrogen-binding domain (EBD) and the other dimerization domain may comprise a streptavidin binding domain; and the CID may be an estrone/biotin fusion protein or a derivative thereof.

One dimerization domain may comprise a glucocorticoid-binding domain (GBD) and the other dimerization domain may comprise a dihydrofolate reductase (DHFR) binding domain; and the CID may be a dexamethasone/methotrexate fusion protein or a derivative thereof.

One dimerization domain may comprise an 06-alkylguanine-DNA alkyltransferase (AGT) binding domain and the other dimerization domain may comprise a dihydrofolate reductase (DHFR) binding domain; and the CID may be an 06-benzylguanine derivative/methotrexate fusion protein or a derivative thereof.

One dimerization domain may comprise a retinoic acid receptor domain and the other dimerization domain may comprise an ecodysone receptor domain; and the CID may be RSL1 or a derivative thereof.

Where the dimerization domain spontaneously heterodimerizes, it may be based on the dimerization domain of an antibody. In particular it may comprise the dimerization portion of a heavy chain constant domain (CH) and a light chain constant domain (CL). The “dimerization portion” of a constant domain is the part of the sequence which forms the inter-chain disulphide bond.

The chimeric cytokine receptor may comprise the Fab portion of an antibody as exodomain, for example as illustrated schematically in FIG. 5.

The chimeric cytokine receptor comprise two polypeptides:
(i) a first polypeptide which comprises:
   (a) a first dimerisation domain; and
   (b) a first chain of the cytokine receptor endodomain; and
(ii) a second polypeptide which comprises:
   (a) a second dimerization domain, which dimerises with the first dimerization domain; and
   (b) a second chain of the cytokine-receptor endodomain.

Cytokine Receptors and Signalling

Many cell functions are regulated by members of the cytokine receptor superfamily. Signalling by these receptors depends upon their association with Janus kinases (JAKs), which couple ligand binding to tyrosine phosphorylation of signalling proteins recruited to the receptor complex. Among these are the signal transducers and activators of transcription (STATs), a family of transcription factors that contribute to the diversity of cytokine responses.

When the chimeric cytokine receptor of the invention binds its ligand or dimerises, one or more of the following intracellular signaling pathways may be initiated:

(i) the JAK-STAT pathway

(ii) the MAP kinase pathway; and

(iii) the Phosphoinositide 3-kinase (PI3K) pathway.

The JAK-STAT system consists of three main components: (1) a receptor (2) Janus kinase (JAK) and (3) Signal Transducer and Activator of Transcription (STAT).

JAKs, which have tyrosine kinase activity, bind to cell surface cytokine receptors. The binding of the ligand to the receptor triggers activation of JAKs. With increased kinase activity, they phosphorylate tyrosine residues on the receptor and create sites for interaction with proteins that contain phosphotyrosine-binding SH2 domains. STATs possessing SH2 domains capable of binding these phosphotyrosine residues are recruited to the receptors, and are themselves tyrosine-phosphorylated by JAKs. These phosphotyrosines then act as binding sites for SH2 domains of other STATs, mediating their dimerization. Different STATs form hetero- or homodimers. Activated STAT dimers accumulate in the cell nucleus and activate transcription of their target genes.

Cytokine Receptor Endodomain

The chimeric cytokine receptor of the present invention comprises an endodomain which causes “cytokine-type” cell signalling (either alone or when in the presence of another chimeric cytokine receptor).

The endodomain may be a cytokine receptor endodomain.

The endodomain may be derived from a type I cytokine receptor. Type I cytokine receptors share a common amino acid motif (WSXWS) in the extracellular portion adjacent to the cell membrane.

The endodomain may be derived from a type I cytokine receptor. Type I cytokine receptors include those that bind type I and type II interferons, and those that bind members of the interleukin-10 family (interleukin-10, interleukin-20 and interleukin-22).

Type I cytokine receptors include:
(i) Interleukin receptors, such as the receptors for IL-2, IL-3, IL-4, IL-5, IL-6, IL-7, IL-9, IL-11, IL-12, IL13, IL-15, IL-21, IL-23 and IL-27;
(ii) Colony stimulating factor receptors, such as the receptors for erythropoietin, GM-CSF, and G-CSF; and
(iii) Hormone receptor/neuropeptide receptor, such as hormone receptor and prolactin receptor

Members of the type I cytokine receptor family comprise different chains, some of which are involved in ligand/cytokine interaction and others that are involved in signal transduction. For example the IL-2 receptor comprises an α-chain, a β-chain and a γ-chain.

The IL-2 receptor common gamma chain (also known as CD132) is shared between the IL-2 receptor, IL-4 receptor, IL-7 receptor, IL-9 receptor, IL-13 receptor and IL-15 receptor.

IL-2

IL-2 binds to the IL-2 receptor, which has three forms, generated by different combinations of three different proteins, often referred to as “chains”: α, β and γ; these subunits are also parts of receptors for other cytokines. The β and γ chains of the IL-2R are members of the type I cytokine receptor family.

The three receptor chains are expressed separately and differently on various cell types and can assemble in different combinations and orders to generate low, intermediate, and high affinity IL-2 receptors.

The α chain binds IL-2 with low affinity, the combination of β and γ together form a complex that binds IL-2 with intermediate affinity, primarily on memory T cells and NK cells; and all three receptor chains form a complex that binds IL-2 with high affinity (Kd˜10-11 M) on activated T cells and regulatory T cells.

The three IL-2 receptor chains span the cell membrane and extend into the cell, thereby delivering biochemical signals to the cell interior. The alpha chain does not participate in signalling, but the beta chain is complexed with the tyrosine phosphatase JAK1. Similarly the gamma chain complexes with another tyrosine kinase called JAK3. These enzymes are activated by IL-2 binding to the external domains of the IL-2R.

IL-2 signalling promotes the differentiation of T cells into effector T cells and into memory T cells when the initial T cells are also stimulated by an antigen. Through their role in the development of T cell immunologic memory, which depends upon the expansion of the number and function of antigen-selected T cell clones, they also have a key role in long-term cell-mediated immunity.

The chimeric cytokine receptor of the present invention may comprise the IL-2 receptor β-chain and/or the IL-2 receptor (i.e. common) γ-chain

The amino acid sequences for the endodomains of the IL-2 β-chain and common γ-chain are shown as SEQ ID No. 1 and 2

SEQ ID No. 1:
Endodomain derived from human common
gamma chain:
ERTMPRIPTLKNLEDLVTEYHGNFSAWSGVSKGLA
ESLQPDYSERLCLVSEIPPKGGALGEGPGASPCNQ
HSPYWAPPCYTLKPET
SEQ ID No. 2:
Endodomain derived from human IL-2Rβ:
NCRNTGPWLKKVLKCNTPDPSKFFSQLSSEHGGDV
QKWLSSPFPSSSFSPGGLAPEISPLEVLERDKVTQ
LLLQQDKVPEPASLSSNHSLTSCFTNQGYFFFHLP
DALEIEACQVYFTYDPYSEEDPDEGVAGAPTGSSP
QPLQPLSGEDDAYCTFPSRDDLLLFSPSLLGGPSP
PSTAPGGSGAGEERMPPSLQERVPRDWDPQPLGPP
TPGVPDLVDFQPPPELVLREAGEEVPDAGPREGVS
FPWSRPPGQGEFRALNARLPLNTDAYLSLQELQGQ
DPTHLV

The term “derived from” means that the endodomain of the chimeric cytokine receptor of the invention has the same sequence as the wild-type sequence of the endogenous molecule, or a variant thereof which retains the ability to form a complex with JAK-1 or JAK-3 and activate one of the signalling pathways mentioned above.

A “variant” sequence having at least 80, 85, 90, 95, 98 or 99% sequence identity to the wild-type sequence (e.g. SEQ ID Nos. 1 or 2), providing that the variant sequence retains the function of the wild-type sequence i.e. the ability to form a complex with JAK-1 or JAK-3 and activate, for example, the JAK-STAT signalling pathway.

The percentage identity between two polypeptide sequences may be readily determined by programs such as BLAST which is freely available at http://blast.ncbi.nlm.nih.gov.

IL-7

The interleukin-7 receptor is made up of two chains: the interleukin-7 receptor-α chain (CD127) and common-γ chain receptor (CD132). The common-γ chain receptors is shared with various cytokines, including interleukin-2, -4, -9, and -15. Interleukin-7 receptor is expressed on various cell types, including naive and memory T cells.

The interleukin-7 receptor plays a critical role in the development of lymphocytes, especially in V(D)J recombination. IL-7R also controls the accessibility of a region of the genome that contains the T-cell receptor gamma gene, by STAT5 and histone acetylation. Knockout studies in mice suggest that blocking apoptosis is an essential function of this protein during differentiation and activation of T lymphocytes.

The chimeric cytokine receptor of the present invention may comprise the IL-7 receptor α-chain and/or the IL-7 receptor (i.e. common) γ-chain, or a variant thereof.

The amino acid sequence for the endodomain of the IL-7 α-chain is shown as SEQ ID No. 3.

SEQ ID No. 3-
Endodomain derived from human IL-7Rα:
KKRIKPIVWPSLPDHKKTLEHLCKKPRKNLNVSFN
PESFLDCQIHRVDDIQARDEVEGFLQDTFPQQLEE
SEKQRLGGDVQSPNCPSEDVVITPESFGRDSSLTC
LAGNVSACDAPILSSSRSLDCRESGKNGPHVYQDL
LLSLGTTNSTLPPPFSLQSGILTLNPVAQGQPILT
SLGSNQEEAYVTMSSFYQNQ

IL-15

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following viral infection. IL-15 induces cell proliferation of natural killer cells.

Interleukin-15 receptor consists of an interleukin 15 receptor alpha subunit and shares common beta and gamma subunits with the IL-2 receptor.

The amino acid sequence for the endodomain of IL-15Rα is shown as SEQ ID No. 60.

SEQ ID No. 60-
Endodomain derived from human IL-15Rα:
SRQTPPLASVEMEAMEALPVTWGTSSRDEDLENCSHHL

Truncated Cytokine Receptor Endodomains

In the chimeric cytokine receptor of the present invention, one or both of the cytokine receptor endodomain chains are truncated. The present inventors have found that it is possible to modulate the activity of the CCR by truncating the C-terminus of one or both chains of the cytokine receptor endodomain.

A schematic diagram illustrating the general structure of a cytokine receptor endodomain is shown in FIG. 11. The endodomain contains elements know as Box 1 and Box 2 which are important for JAK binding. A series of tyrosine residues are present on the intracellular part which are phosphorylated upon receptor activation.

The sequence of the endodomain derived from human IL-2Rβ is shown above as SEQ ID No. 2. The Box 1 motif is from amino acids 278-286 in the full length sequence and has the sequence KCNTPDPS (SEQ ID No. 47). The Box 2 motif is from amino acids 323-333 in the full length sequence and has the sequence SPLEVLERDKV (SEQ ID No. 48).

IL2BR endodomain, showing Box 1 and Box 2 and
tyrosine residues
(SEQ ID No. 2)
NCRNTGPWLKKVL [symbol] KFFSQLSSEHGGDVQKWLSSPFPS
SSFSPGGLAPEI [symbol] TQLLLQQDKVPEPASLSSNHS
LTSCFTNQG [symbol] FFFHLPDALEIEACQV [symbol] FT [symbol] DP [symbol] SEEDPDEGVAG
APTGSSPQPLQPLSGEDDA [symbol] CTFPSRDDLLLFSPSLLGGPSPPST
APGGSGAGEERMPPSLQERVPRDWDPQPLGPPTPGVPDLVDFQPP
PELVLREAGEEVPDAGPREGVSFPWSRPPGQGEFRALNARLPLNT
DA [symbol] LSLQELQGQDPTHLV

Where the CCR of the present invention comprises a receptor endodomain from a type I the type I cytokine receptor family, it may comprise an endodomain which is truncated at the C-terminus but which retains the Box 1 and Box 2 motif.

The endodomain from human IL-2Rβ is 286 amino acids in length, as shown in SEQ ID No. 2 and FIG. 10a. A truncated version of IL-2Rβ may lack up to 218 amino acids from the C-terminus. This means that the Box-1 and Box 2 motifs will be retained, as they are in the first 68 amino acid of the sequence. A truncated version of IL-2Rβ may have a C-terminal truncation of up to 200 amino acids, up to 180 amino acids, up to 160 amino acids, up to 140 amino acids, up to 120 amino acids, up to 100 amino acids, up to 80 amino acids, up to 60 amino acids, up to 40 amino acids, or up to 20 amino acids.

A truncated version of IL-2Rβ may have a truncation of between 10 and 200 amino acids, between 20 and 180 amino acids, between 40 and 180, between 60 and 160, between 80 and 140 or between 100 and 120 amino acids. As shown in FIG. 10b, a truncation of between 40 amino acids (i.e. IL2Rbeta aa 266-511) and 180 amino acids (i.e. IL2Rbeta aa 266-371) gives a progressive reduction in cytokine signalling activity, so it is possible to “tune down” the cytokine signal by choosing a deletion in the range.

A truncated version of IL-2Rβ may have one of the sequences shown as SEQ ID No. 49 to 59. A truncated version of IL-2Rβ may have a sequence “between” two of the truncated sequences shown as SEQ ID No. 49 to 59, for example, a sequence “between II2Rbeta aa266-411 (SEQ ID NO. 53) and II2Rbeta aa266-431 (SEQ ID NO. 54) may be aa266-412, aa266-413, etc. . . . until aa266-429, aa266-430.

II2Rbeta aa266-331 (SEQ ID NO. 49):
NCRNTGPWLKKVLKCNTPDPSKFFSQLSSEHGGDVQKWLSSPFPSSS
FSPGGLAPEISPLEVLERDKV
II2Rbeta aa266-351 (SEQ ID NO. 50):
NCRNTGPWLKKVLKCNTPDPSKFFSQLSSEHGGDVQKWLSSPFPSSS
FSPGGLAPEISPLEVLERDKVTQLLLQQDKVPEPASLSS
II2Rbeta aa266-371 (SEQ ID NO. 51):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLP
II2Rbeta aa266-391 (SEQ ID NO. 52):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEED
II2Rbeta aa266-411 (SEQ ID NO. 53):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPL
II2Rbeta aa266-431 (SEQ ID NO. 54):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFS
II2Rbeta aa266-451 (SEQ ID NO. 55):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAG
II2Rbeta aa266-471 (SEQ ID NO. 56):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEE
RMPPSLQERVPRDWDPQP
II2Rbeta aa266-491 (SEQ ID NO. 57):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEE
RMPPSLQERVPRDWDPQPLGPPTPGVPDLVDFQPPPELV
II2Rbeta aa266-511 (SEQ ID NO. 58):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEE
RMPPSLQERVPRDWDPQPLGPPTPGVPDLVDFQPPPELVLREAGEEV
PDAGPREGVSF
II2Rbeta aa266-531 (SEQ ID NO. 59):
NCRNTGPWLKKVLKCNTPDPSKFFSQLSSEHGGDVQKWLSSPFPSSS
FSPGGLAPEISPLEVLERDKVTQLLLQQDKVPEPASLSSNHSLTSCF
TNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEE
RMPPSLQERVPRDWDPQPLGPPTPGVPDLVDFQPPPELVLREAGEEV
PDAGPREGVSFPWSRPPGQGEFRALNARLPL

As shown in FIG. 11 and the annotated version of SEQ ID No. 2 above, IL-2Rβ has six tyrosine residues in the endodomain. Tyrosine residues become phosphorylated upon receptor activation. A truncated version of IL-2Rβ may lack one or more tyrosine residues in its endodomain, compared to the wild-type sequence. A truncated version of IL-2Rβ endodomain may lack 1, 2, 3, 4, 5 or all 6 tyrosine residues compared to the wild-type sequence.

The endodomain derived from human common gamma chain has the sequence shown above as SEQ ID No. 1 above, which has 86 amino acids. A truncated version of this sequence may, for example, have a C-terminal truncation of up to 60, up to 50, up to 40, up to 30, up to 20 or up to 10 amino acids.

The common gamma chain has four tyrosine residues in the endodomain. A truncated version of common gamma chain endodomain may lack one or more tyrosine residues compared to the wild-type sequence. A truncated version of common gamma chain endodomain may lack 1, 2, 3, or all 4 tyrosine residues compared to the wild-type sequence.

The endodomain derived from human IL-7Rα has the sequence shown above as SEQ ID No. 3 above, which has 195 amino acids. A truncated version of this sequence may, for example, have a C-terminal truncation of up to 120, up to 100, up to 80, up to 60, up to 40 or up to 20 amino acids.

Human IL-7Rα has three tyrosine residues in the endodomain. A truncated version of human IL-7Rα endodomain may lack one or more tyrosine residues compared to the wild-type sequence. A truncated version of human IL-7Rα endodomain may lack 1, 2, or all 3 tyrosine residues compared to the wild-type sequence.

The endodomain of IL-15Rα is shown above as SEQ ID No. 60 and has 38 amino acids. A truncated version of this sequence may, for example, have a C-terminal truncation of up to 20, up to 15, up to 10, or up to 5 amino acids.

For any given cytokine receptor endodomain, a truncated version for use in the present invention may have a C-terminal deletion of up to 60%, up to 50%, up to 40%, up to 30%, up to 20% or up to 10% of the amino acids compared to the wild-type endodomain sequence. The deletion may be between 10 and 60%, 20 and 50%, or 30 and 40%.

In the chimeric cytokine receptor, one or more chains may have a truncated sequence. For example, in a CCR with and IL-2 receptor endodomain comprising the IL-2 receptor β-chain and/or the IL-2 receptor (i.e. common) γ-chain, the IL-2 receptor β-chain and/or the common γ-chain may be truncated.

Method for Modulating Activity of a CCR

The present invention also provides a method for modulating the activity of a chimeric cytokine receptor (CCR) by truncating one or more chains in the cytokine receptor endodomain. Activity of the CCR, and therefore cytokine signalling, may be increased or decreased, depending on the truncation.

As shown in Example 5, it is possible to ascertain the effect of truncating cytokine receptor endodomains in CCRs by preparing constructs with a panel of deletions and investigating the effect on cytokine signalling by looking at a parameter such as cell proliferation.

It is also possible to tailor cytokine signalling to the desired level by choosing a cytokine receptor endodomain truncation or combination of truncations which gives the desired level of activity in terms of cytokine signalling mediated by the CCR.

The present invention also provides a method for reducing the activity of a chimeric cytokine receptor (CCR) by truncating one or more chains in the cytokine receptor endodomain.

For example, the activity of a CCR containing an IL-2 receptor beta endodomain can be reduced by truncating the IL-2Rbeta between 40 and 180 amino acids at the C-terminus.

Spacer

The chimeric cytokine receptor of the present invention may comprise a spacer to connect the antigen-binding domain or dimerization domain with the transmembrane domain and spatially separate the antigen-binding domain or dimerization domain from the endodomain. A flexible spacer allows to an antigen-binding domain to orient in different directions to enable antigen binding.

Where the cell of the present invention comprises two or more chimeric cytokine receptors, the spacers may be the same or different. Where the cell of the present invention comprises a chimeric cytokine receptor (CCR) and a chimeric antigen receptor (CAR), the spacer of the CCR and the CAR may be different, for example, having a different length. The spacer of the CAR may be longer than the spacer of the or each CCR.

The spacer sequence may, for example, comprise an IgG1 Fc region, an IgG1 hinge or a CD8 stalk. The linker may alternatively comprise an alternative linker sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

(hinge-CH2CH3 of human IgG1)
SEQ ID No. 4
AEPKSPDKTHTCPPCPAPPVAGPSVFLFPPKPKDTLMIARTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRD
ELTKNQVSLTCLVKGFYPSDIAVEWESNGQPENNYKTTPPVLDSDGS
FFLYSKLTVDKSRWQQGNVFSCSVMHEALHNHYTQKSLSLSPGKKD
(human CD8 stalk):
SEQ ID No. 5
TTTPAPRPPTPAPTIASQPLSLRPEACRPAAGGAVHTRGLDFACDI
(human IgG1 hinge):
SEQ ID No. 6
AEPKSPDKTHTCPPCPKDPK

Transmembrane Domain

The transmembrane domain is the sequence of a CCR that spans the membrane. It may comprise a hydrophobic alpha helix. The transmembrane domain may be derived from CD28, which gives good receptor stability.

Alternatively the transmembrane domain may be derived from a cytokine receptor, for example the same cytokine from which the endodomain is derived.

The transmembrane domain may, for example be derived from IL-2R, IL-7R or IL-15R.

Transmembrane derived from human common
gamma chain:
SEQ ID No. 7
VVISVGSMGLIISLLCVYFWL
Transmembrane derived from human IL-2Rβ:
SEQ ID No. 8
IPWLGHLLVGLSGAFGFIILVYLLI
Transmembrane derived from human IL-7Rα:
SEQ ID No. 9
PILLTISILSFFSVALLVILACVLW
Transmembrane derived from human IL-15Rα:
SEQ ID No. 10
AISTSTVLLCGLSAVSLLACYL

Ligand-Binding Exodomain

The ligand binding domain comprises an antigen binding domain. The antigen binding domain binds the target ligand for the CCR, i.e. the tumour secreted factor or chemokine or cell surface antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; the binding domain from a natural receptor for the target antigen; a peptide with sufficient affinity for the target ligand; a single domain binder such as a camelid; an artificial binder single as a Darpin; or a single-chain derived from a T-cell receptor.

The term “ligand” is used synonymously with “antigen” to mean an entity which is specifically recognised and bound by the antigen-binding domain of the CCR.

Where the ligand is a tumour secreted factor, the antigen binding domain may comprise an immunoglobulin-based antigen binding site, such as an scFv or a single domain binder.

Where the ligand is a chemokine, the antigen binding domain may comprise the chemokine-binding portion of a natural receptor for the chemokine.

Ligand

The CCR of the present invention may bind a ligand.

The ligand may be a soluble ligand such as a tumour secreted factor or a chemokine.

Alternatively, the ligand may be a membrane bound ligand, such as a cell surface antigen.

The term “soluble ligand” is used to indicate a ligand or antigen which is not part of or attached to a cell but which moves freely in the extracellular space, for example in a bodily fluid of the tissue of interest. The soluble ligand may exist in a cell-free state in the serum, plasma or other bodily fluid of an individual.

The soluble ligand may be associated with the presence or pathology of a particular disease, such as cancer.

The soluble ligand may be part of the cancer secretome, i.e. the collection of factors secreted by a tumour, be it from cancer stem cells, non-stem cells or the surrounding stroma. The soluble ligand may be secreted or shed by tumour cells (see next section).

The soluble ligand may be characteristic of a disease or of diseased tissue. It may be found exclusively, or at a higher level in a subject having the disease vs a healthy subject; or in diseased tissue vs healthy tissue. The soluble ligand may be expressed at at least a 2-fold, 5-fold, 10-fold, 100-fold, 1000-fold, 10,000-fold or 100,000 fold higher level a subject having the disease vs a healthy subject; or in diseased tissue vs healthy tissue.

The terms “cell-surface antigen” and “cell-surface ligand” is used synonymously with “membrane-bound antigen” and “membrane-bound ligand” to mean a ligand which is attached to or expressed on the surface of the cell. The cell-surface ligand may, for example, be a transmembrane protein.

The cell on which the cell-surface ligand is found may be a target cell, such as a cancer cell.

The cell-surface ligand may be associated with the presence or pathology of a particular disease, such as cancer. Alternatively the cell-surface ligand may be characteristic of the cell type of the target cell (e.g. B-cell) without being necessarily associated with the diseased state.

Where the cell-surface ligand is characteristic of a disease or of diseased tissue it may be found exclusively, or at a higher level on the relevant cells a subject having the disease vs a healthy subject; or in diseased tissue vs healthy tissue. The cell-surface ligand may be expressed at at least a 2-fold, 5-fold, 10-fold, 100-fold, 1000-fold, 10,000-fold or 100,000 fold higher level on a cell of a subject having the disease vs a healthy subject; or in diseased tissue vs healthy tissue.

Tumour Secreted Factor

The ligand recognised by the CCR may be a soluble ligand secreted by or shedded from a tumour.

This “tumour secreted factor” may, for example, be prostate-specific antigen (PSA), carcinoembryonic antigen (CEA), vascular endothelial growth factor (VEGF) or Cancer Antigen-125 (CA-125).

The tumour secreted factor may be a soluble ligand which is not a cytokine. The CCR of the present invention therefore grafts the binding specificity for a non-cytokine ligand on to the endodomain of a cytokine receptor.

Prostate-Specific Antigen (PSA)

The soluble ligand may be prostate-specific antigen (PSA).

Prostate-specific antigen (PSA), also known as gamma-seminoprotein or kallikrein-3 (KLK3), is a glycoprotein enzyme encoded in humans by the KLK3 gene. PSA is a member of the kallikrein-related peptidase family and is secreted by the epithelial cells of the prostate gland.

PSA is present in small quantities in the serum of men with healthy prostates, but is elevated in individuals with prostate cancer and other prostate disorders.

PSA is a 237-residue glycoprotein and is activated by KLK2. Its physiological role is the liquefaction of the coagulum components of the semen leading to liberation of spermatozoa. In cancer, PSA may participate in the processes of neoplastic growth and metastasis.

PSA is a chymotrypsin-like serine protease with a typical His-Asp-Ser triad and a catalytic domain similar to those of other kallikrein-related peptidases. The crystal structure of PSA has been obtained i) in complex with the monoclonal antibody (mAb) 8G8F5 and ii) in a sandwich complex with two mAbs 5D5A5 and 5D3D11 (Stura et al (J. Mol. Biol. (2011) 414:530-544).

Various monoclonal antibodies are known, including clones 2G2-B2, 2D8-E8, IgG1/K described in Bavat et al Avicenna J. Med. Biotechnol. 2015, 7:2-7; and Leinonen (2004) 289:157-67.

The CCR of the present invention may, for example, comprise the 6 CDRs or the VH and/or VL domain(s) from a PSA-binding mAb such as 8G8F5, 5D5A5 or 5D3D11.

Where the CCR comprises two antigen binding specificities, binding different epitopes on PSA, one may be based on, for example 5D3D11 and one may be based on, for example, 5D5A5.

The amino acid sequences for 5D3D11 and 5D5A5 VH and VL are given below. The complementarity determining regions (CDRs) are highlighted in bold.

5D3D11 VH
(SEQ ID No. 11)
QVQLQQSGPELVKPGASVKISCKVSGYAIS [symbol] WVKQRPGQGLEW
IG [symbol] [symbol] KATLTVDKSSSTAYMQLSSLTSVDSAV
YFCAR [symbol] WGQGTSVTVSS
5D3D11 VL
(SEQ ID No. 12)
DIVMTQTAPSVFVTPGESVSISC [symbol] WFLQRPGQ
SPQLLIY [symbol] [symbol] GVPDRFSGSGSGTDFTLRISRVEAEDVGVYYC [symbol]
FGAGTKVEIK
5D5A5 VH
(SEQ ID No. 13)
QVQLQQSGAELAKPGASVKMSCKTSGYSFS [symbol] WVKQRPGQGLEW
IG [symbol] [symbol] KVTLTADKSSNTAYMQLNSLTSEDSAVY
YCAR [symbol] WGAGTTVTVSS
5D5A5 VL
(SEQ ID No. 14)
DIVLTQSPPSLAVSLGQRATISC [symbol] WYQQKPGQP
PKILIY [symbol] [symbol] GIPARFSGSGSRTDFTLTINPVEADDVATYYC [symbol]
FGGGTKLEIK
ScFv based on 5D5A5
(SEQ ID No. 15)
QVQLQQSGAELAKPGASVKMSCKTSGYSFSSYWMHWVKQRPGQGLEW
IGYINPSTGYTENNQKFKDKVTLTADKSSNTAYMQLNSLTSEDSAVY
YCARSGRLYFDVWGAGTTVTVSSGGGGSGGGGSGGGGSGGGGSDIVL
TQSPPSLAVSLGQRATISCRASESIDLYGFTFMHWYQQKPGQPPKIL
IYRASNLESGIPARFSGSGSRTDFTLTINPVEADDVATYYCQQTHED
PYTFGGGTKLEIK
ScFv based on 5D3D11
(SEQ ID No. 16)
QVQLQQSGPELVKPGASVKISCKVSGYAISSSWMNWVKQRPGQGLEW
IGRIYPGDGDTKYNGKFKDKATLTVDKSSSTAYMQLSSLTSVDSAVY
FCARDGYRYYFDYWGQGTSVTVSSGGGGSGGGGSGGGGSGGGGSDIV
MTQTAPSVFVTPGESVSISCRSSKSLLHSNGNTYLYWFLQRPGQSPQ
LLIYRMSNLASGVPDRFSGSGSGTDFTLRISRVEAEDVGVYYCMQHL
EYPVTFGAGTKVEIK

Where a cell comprises two CCRs, the antigen-binding domain of the first CCR may comprise the 6 CDRs from 5D5A5 and the antigen-binding domain of the second CCR may comprise the 6 CDRs from 5D3D11.

The antigen-binding domain of the first CCR may comprise the VH and/or VL domain(s) from 5D5A5 or a variant thereof; and the antigen-binding domain of the second CCR may comprise the VH and/or VL domain(s) from 5D3D11 or a variant thereof. Variant VH and VL domains may have at least 80, 90, 95 or 99% identity to the sequences given above, provided that they retain PSA-binding activity.

A cell expressing a CCR which binds PSA may be useful in the treatment of prostate cancer.

Carcinoembryonic Antigen (CEA)

The soluble ligand may be CEA.

Carcinoembryonic antigen (CEA) describes a set of highly related glycoproteins involved in cell adhesion. CEA is normally produced in gastrointestinal tissue during fetal development, but the production stops before birth. Therefore CEA is usually present only at very low levels in the blood of healthy adults. However, the serum levels are raised in some types of cancer, which means that it can be used as a tumor marker in clinical tests.

CEA are glycosyl phosphatidyl inositol (GPI) cell surface anchored glycoproteins whose specialized sialofucosylated glycoforms serve as functional colon carcinoma L-selectin and E-selectin ligands, which may be critical to the metastatic dissemination of colon carcinoma cells. Immunologically they are characterized as members of the CD66 cluster of differentiation.

CEA and related genes make up the CEA family belonging to the immunoglobulin superfamily. In humans, the carcinoembryonic antigen family consists of 29 genes, 18 of which are normally expressed. The following is a list of human genes which encode carcinoembryonic antigen-related cell adhesion proteins: CEACAM1, CEACAM3, CEACAM4, CEACAM5, CEACAM6, CEACAM7, CEACAM8, CEACAM16, CEACAM18, CEACAM19, CEACAM20, CEACAM21 Various antibodies which target CEA are described in WO 2011/034660.

A cell expressing a CCR against CEA may be useful in the treatment of, for example, colorectal cancer.

Vascular Endothelial Growth Factor (VEGF)

The soluble ligand may be VEGF.

Vascular endothelial growth factor (VEGF) is a signal protein produced by cells that stimulates vasculogenesis and angiogenesis. It is part of the system that restores the oxygen supply to tissues when blood circulation is inadequate. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

When VEGF is overexpressed, it can contribute to disease. Solid cancers cannot grow beyond a limited size without an adequate blood supply; cancers that can express VEGF are able to grow and metastasize.

VEGF is a sub-family of the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature).

The VEGF family comprises in mammals five members: VEGF-A, placenta growth factor (PGF), VEGF-B, VEGF-C and VEGF-D.

Various antibodies to VEGF are known, such as bevacizumab (Avastin) and Ranibizumab (Lucentis).

Cancer Antigen 125 (CA-125)

CA-125 is associated with ovarian cancer and is the most frequently used biomarker for ovarian cancer detection. While CA-125 is best known as a marker for ovarian cancer, it may also be elevated in other cancers, including endometrial cancer, fallopian tube cancer, lung cancer, breast cancer and gastrointestinal cancer.

The sequence of human CA-125 (also known as mucin-16) is available from NCBI, Accession No. 078966.

A number of CA125-binding monoclonal antibodies are known, including OC125 and M11 (Nustad et al 1996, Tumour Biol. 17:196-329). In this study the specificity of 26 monoclonal antibodies against the CA 125 antigen was investigated. It was found that the CA 125 antigen carries only two major antigenic domains, which classifies the antibodies as OC125-like (group A) or M11-like (group B).

The chimeric cytokine receptor of the present invention may comprise an antigen-binding domain from such an antibody. A cell comprising such a CCR may be useful in the treatment of, for example, ovarian cancer.

The tumour secreted factor (or, if in a membrane-bound form, the transmembrane protein) may be selected from the following non-exhaustive list:

ALK gene rearrangements and overexpression giving mutated forms of ALK proteins

Alpha-fetoprotein (AFP)

Beta-2-microglobulin (B2M)

Beta-human chorionic gonadotropin (Beta-hCG)

BRAF V600 mutations giving mutated B-REF protein

C-kit/CD117

CA15-3/CA27.29

CA19-9

Calcitonin

CD20

Chromogranin A (CgA)

Cytokeratin fragment 21-1

EGFR gene mutation analysis

Estrogen receptor (ER)/progesterone receptor (PR)

Fibrin/fibrinogen

HE4

HER2/neu gene amplification or protein overexpression

Immunoglobulins

KRAS gene mutation analysis

Lactate dehydrogenase

Neuron-specific enolase (NSE)

Nuclear matrix protein 22

Programmed death ligand 1 (PD-L1)

Thyroglobulin

Urokinase plasminogen activator (uPA) and plasminogen activator inhibitor (PAI-1)

Chemokine

Chemokines are chemotactic cytokines. Cell migration is guided by chemokine gradients embedded and immobilized in extracellular matrix. The positively charged chemokines like CXCL12 bind to negatively charged ECM molecules. These gradients provide tracks for cancer cell and immune cell homing. The action on T cells seems to be inhibitory for the homing of cytotoxic T cells, while regulatory T cells appear to be attracted.

Chemokines are approximately 8-10 kilodaltons in mass and have four cysteine residues in conserved locations which are key to forming their 3-dimensional shape.

Some chemokines are considered pro-inflammatory and can be induced during an immune response to recruit cells of the immune system to a site of infection, while others are considered homeostatic and are involved in controlling the migration of cells during normal processes of tissue maintenance or development.

Chemokines have been classified into four main subfamilies: CXC, CC, CX3C and XC. All of these proteins exert their biological effects by interacting with G protein-linked transmembrane receptors called chemokine receptors that are selectively found on the surfaces of their target cells.

The major role of chemokines is to act as a chemoattractant to guide the migration of cells. Cells that are attracted by chemokines follow a signal of increasing chemokine concentration towards the source of the chemokine. Some chemokines control cells of the immune system during processes of immune surveillance, such as directing lymphocytes to the lymph nodes so they can screen for invasion of pathogens by interacting with antigen-presenting cells residing in these tissues. Other chemokines are inflammatory and are released from a wide variety of cells in response to bacterial infection, viruses and other agents. Their release is often stimulated by pro-inflammatory cytokines such as interleukin 1. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing. They are released by many different cell types and serve to guide cells of both innate immune system and adaptive immune system.

CC Chemokines

The CC chemokine (or β-chemokine) proteins have two adjacent cysteines (amino acids), near their amino terminus. There have been at least 27 distinct members of this subgroup reported for mammals, called CC chemokine ligands (CCL)-1 to -28; CCL10 is the same as CCL9. Chemokines of this subfamily usually contain four cysteines (C4-CC chemokines), but a small number of CC chemokines possess six cysteines (C6-CC chemokines). C6-CC chemokines include CCL1, CCL15, CCL21, CCL23 and CCL28. CC chemokines induce the migration of monocytes and other cell types such as NK cells and dendritic cells.

Examples of CC chemokine include monocyte chemoattractant protein-1 (MCP-1 or CCL2) which induces monocytes to leave the bloodstream and enter the surrounding tissue to become tissue macrophages.

CCL5 (or RANTES) attracts cells such as T cells, eosinophils and basophils that express the receptor CCR5.

CXC Chemokines

The two N-terminal cysteines of CXC chemokines (or α-chemokines) are separated by one amino acid, represented in this name with an “X”. There have been 17 different CXC chemokines described in mammals, that are subdivided into two categories, those with a specific amino acid sequence (or motif) of glutamic acid-leucine-arginine (or ELR for short) immediately before the first cysteine of the CXC motif (ELR-positive), and those without an ELR motif (ELR-negative). ELR-positive CXC chemokines specifically induce the migration of neutrophils, and interact with chemokine receptors CXCR1 and CXCR2.

C Chemokines

The third group of chemokines is known as the C chemokines (or γ chemokines), and is unlike all other chemokines in that it has only two cysteines; one N-terminal cysteine and one cysteine downstream. Two chemokines have been described for this subgroup and are called XCL1 (lymphotactin-α) and XCL2 (lymphotactin-β).

CX3C Chemokine

CX3C chemokines have three amino acids between the two cysteines. The only CX3C chemokine discovered to date is called fractalkine (or CX3CL1). It is both secreted and tethered to the surface of the cell that expresses it, thereby serving as both a chemoattractant and as an adhesion molecule.

Chemokine receptors are G protein-coupled receptors containing 7 transmembrane domains that are found on the surface of leukocytes. Approximately 19 different chemokine receptors have been characterized to date, which are divided into four families depending on the type of chemokine they bind; CXCR that bind CXC chemokines, CCR that bind CC chemokines, CX3CR1 that binds the sole CX3C chemokine (CX3CL1), and XCR1 that binds the two XC chemokines (XCL1 and XCL2). They share many structural features; they are similar in size (with about 350 amino acids), have a short, acidic N-terminal end, seven helical transmembrane domains with three intracellular and three extracellular hydrophilic loops, and an intracellular C-terminus containing serine and threonine residues important for receptor regulation. The first two extracellular loops of chemokine receptors each has a conserved cysteine residue that allow formation of a disulfide bridge between these loops. G proteins are coupled to the C-terminal end of the chemokine receptor to allow intracellular signaling after receptor activation, while the N-terminal domain of the chemokine receptor determines ligand binding specificity.

CXCL12

CXCL12 is strongly chemotactic for lymphocytes. CXCL12 plays an important role in angiogenesis by recruiting endothelial progenitor cells (EPCs) from the bone marrow through a CXCR4 dependent mechanism. It is this function of CXCL12 that makes it a very important factor in carcinogenesis and the neovascularisation linked to tumour progression. CXCL12 also has a role in tumour metastasis where cancer cells that express the receptor CXCR4 are attracted to metastasis target tissues that release the ligand, CXCL12.

The receptor for CXCL12 is CXCR4. The CCR of the present invention may comprise the CXCL12-binding domain from CXCR4 linked to an endodomain derived from a cytokine receptor, such as the IL-2 receptor.

CXCR4 coupled expression of IL2 would support engraftment of therapeutic T cell for cancer therapies. In multiple myeloma, a cell expressing such a CCR may mobilize cells and change the bone marrow environment. Such cells also have uses in the treatment of solid cancers by modifying the solid tumour microenvironment.

The amino acid sequence for CXCR4 is shown below as SEQ ID No. 17

```
SEQ ID No. 17
    1 msiplpllqi ytsdnyteem gsgdydsmke pcfreenanf nkiflptiys iifltgivgn
  61 glvilvmgyq kklrsmtdky rlhlsvadll fvitlpfwav davanwyfgn flckavhviy
121 tvnlyssvli lafisldryl aivhatnsqr prkllaekvv yvgvwipall ltipdfifan
181 vseaddryic drfypndlwv vvfqfqhimv glilpgivil scyciiiskl shskghqkrk
241 alkttvilil affacwlpyy igisidsfil leiikqgcef entvhkwisi tealaffhcc
301 lnpilyaflg akfktsaqha ltsvsrgssl kilskgkrgg hssysteses ssfhss
```

CXCR7 also binds CXCL12.

CCL2

The chemokine (C-C motif) ligand 2 (CCL2) is also referred to as monocyte chemotactic protein 1 (MCP1) and small inducible cytokine A2. CCL2 recruits monocytes, memory T cells, and dendritic cells to the sites of inflammation produced by either tissue injury or infection.

CCR2 and CCR4 are two cell surface receptors that bind CCL2.

CCR2 has the amino acid sequence shown as SEQ ID No. 18

```
SEQ ID No. 18
    1 mlstsrsrfi rntnesgeev ttffdydyga pchkfdvkqi gaqllpplys lvfifgfvgn
  61 mlvvlilinc kklkcltdiy llnlaisdll flitlplwah saanewvfgn amcklftgly
121 higyfggiff iilltidryl aivhavfalk artvtfgvvt svitwlvavf asvpgiiftk
181 cqkedsvyvc gpyfprgwnn fhtimrnilg lvlpllimvi cysgilktll rcrnekkrhr
241 avrviftimi vyflfwtpyn ivillntfqe ffglsncest sqldqatqvt etlgmthcci
301 npiiyafvge kfrslfhial gcriaplqkp vcggpgvrpg knvkvttqgl ldgrgkgksi
361 grapeaslqd kega
```

CCR4 has the amino acid sequence shown as SEQ ID No. 19.

```
SEQ ID No. 19
    1 mnptdiadtt ldesiysnyy lyesipkpct kegikafgel flpplyslvf vfgllgnsvv
  61 vlvlfkykrl rsmtdvylln laisdllfvf slpfwgyyaa dqwvfglglc kmiswmylvg
121 fysgiffvml msidrylaiv havfslrart ltygvitsla twsvavfasl pgflfstcyt
181 ernhtycktk yslnsttwkv lssleinilg lviplgimlf cysmiirtlq hcknekknka
241 vkmifavvvl flgfwtpyni vlfletivel evlqdctfer yldyaiqate tlafvhccln
301 piiyfflgek frkyilqlfk tcrglfvlcq ycgllqiysa dtpsssytqs tmdhdlhdal
```

The CCR of the present invention may comprise the CCL2 binding site of CCR2 or CCR4 in its ligand binding domain.

Cell-Surface Antigen

The ligand may be a cell-surface antigen, such as a transmembrane protein.

The cell surface antigen may be CD22.

CD22, or cluster of differentiation-22, is a molecule belonging to the SIGLEC family of lectins. It is found on the surface of mature B cells and to a lesser extent on some immature B cells. Generally speaking, CD22 is a regulatory molecule that prevents the overactivation of the immune system and the development of autoimmune diseases.

CD22 is a sugar binding transmembrane protein, which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signalling.

Increased expression of CD22 is seen in non-Hodgkin and other lymphomas. Various monoclonal antibodies targeting CD22 are known, including epratuzumab, inotuzumab ozogamicin, m971 and m972.

Chimeric Antigen Receptors (CAR)

The cell of the present invention may also comprise one or more chimeric antigen receptor(s). The CAR(s) may be specific for a tumour-associated antigen.

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like or ligand-based antigen binding site. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

The cell of the present invention may comprise one or more CAR(s).

The CAR(s) may comprise an antigen-binding domain, a spacer domain, a transmembrane domain and an endodomain. The endodomain may comprise or associate with a domain which transmit T-cell activation signals.

Car Antigen Binding Domain

The antigen-binding domain is the portion of a CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

The term “ligand” is used synonymously with “antigen” to mean an entity which is specifically recognised and bound by the antigen-binding domain of a CAR.

Cell Surface Antigen

The CAR may recognise a cell-surface antigen, i.e. an entity, such as a transmembrane protein which is expressed on the surface of a target cell, such as a tumour cell.

The CAR may specifically bind a tumour-associated cell-surface antigen.

Various tumour associated antigens (TAA) are known, some of which are shown in Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Where the CAR recognises a B-cell lymphoma or leukemia antigen (such as CD19, CD20, CD52, CD160 or CD5), the CCR may recognise another B-cell antigen, such as CD22.

Prostate-Cancer Associated Antigens

The CAR may specifically bind a cell-surface antigen associated with prostate cancer, such as prostate stem cell antigen (PSCA) or prostate-specific membrane antigen (PSMA).

PSCA is a glycosylphosphatidylinositol-anchored cell membrane glycoprotein. It is up-regulated in a large proportion of prostate cancers and is also detected in cancers of the bladder and pancreas.

Various anti-PSCA antibodies are known, such as 7F5 (Morgenroth et al (Prostate (2007) 67:1121-1131); 1G8 (Hillerdal et al (2014) BMC Cancer 14:30); and Ha1-4.117 (Abate-Daga et al (2014) 25:1003-1012).

The CCR-expressing cell of the invention may also express an anti-PSCA CAR which may comprise an antigen binding domain based on one of these antibodies.

PSMA is a zinc metalloenzyme that resides in membranes. PSMA is strongly expressed in the human prostate, being a hundredfold greater than the expression in most other tissues. In cancer, it is upregulated in expression and has been called the second-most-upregulated gene in prostate cancer, with increase of 8- to 12-fold over the noncancerous prostate. In addition to the expression in the human prostate and prostate cancer, PSMA is also found to be highly expressed in tumor neovasculature but not normal vasculature of all types of solid tumors, such as kidney, breast, colon, etc.

Various anti-PSMA antibodies are known, such as 7E11, J591, J415, and Hybritech PEQ226.5 and PM2J004.5 each of which binds a distinct epitope of PSMA (Chang et al (1999) Cancer Res 15:3192-8).

The CCR-expressing cell of the invention may also express an anti-PSMA CAR which may comprise an antigen binding domain based on one of these antibodies.

For example, the CCR may comprise an scFv based on J591, having the sequence shown as SEQ ID No. 20.

(J591 scFv)
SEQ ID No. 20
EVQLQQSGPELKKPGTSVRISCKTSGYTFTEYTIHWVKQSHGKSLEW
IGNINPNNGGTTYNQKFEDKATLTVDKSSSTAYMELRSLTSEDSAVY
YCAAGWNFDYWGQGTTLTVSSGGGGSGGGGSGGGGSDIVMTQSHKFM
STSVGDRVSIICKASQDVGTAVDWYQQKPGQSPKLLIYWASTRHTGV
PDRFTGSGSGTDFTLTITNVQSEDLADYFCQQYNSYPLTFGAGTMLD
LKR

CAR Transmembrane Domain

The transmembrane domain is the sequence of a CAR that spans the membrane. It may comprise a hydrophobic alpha helix. The CAR transmembrane domain may be derived from CD28, which gives good receptor stability.

CAR Signal Peptide

The CAR and CCR described herein may comprise a signal peptide so that when it/they is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the sequence shown as SEQ ID No. 21, 22 or 23 or a variant thereof having 5, 4, 3, 2 or 1 amino acid mutations (insertions, substitutions or additions) provided that the signal peptide still functions to cause cell surface expression of the CAR.

SEQ ID No. 21:
MGTSLLCWMALCLLGADHADG

The signal peptide of SEQ ID No. 21 is compact and highly efficient and is derived from TCR beta chain. It is predicted to give about 95% cleavage after the terminal glycine, giving efficient removal by signal peptidase.

SEQ ID No. 22:
MSLPVTALLLPLALLLHAARP

The signal peptide of SEQ ID No. 22 is derived from IgG1.

SEQ ID No. 23:
MAVPTQVLGLLLLWLTDARC

The signal peptide of SEQ ID No. 23 is derived from CD8a.

CAR Endodomain

The endodomain is the portion of a classical CAR which is located on the intracellular side of the membrane.

The endodomain is the signal-transmission portion of a classical CAR. After antigen recognition by the antigen binding domain, individual CAR molecules cluster, native CD45 and CD148 are excluded from the synapse and a signal is transmitted to the cell.

The CAR endodomain may be or comprise an intracellular signalling domain. In an alternative embodiment, the endodomain of the present CAR may be capable of interacting with an intracellular signalling molecule which is present in the cytoplasm, leading to signalling.

The intracellular signalling domain or separate intracellular signalling molecule may be or comprise a T cell signalling domain.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The CAR may comprise the CD3-Zeta endodomain alone, the CD3-Zeta endodomain with that of either CD28 or OX40 or the CD28 endodomain and OX40 and CD3-Zeta endodomain.

The CAR endodomain may comprise one or more of the following: an ICOS endodomain, a CD27 endodomain, a BTLA endodomain, a CD30 endodomain, a GITR endodomain and an HVEM endodomain.

The endodomain may comprise the sequence shown as SEQ ID No. 24 to 32 or a variant thereof having at least 80% sequence identity.

CD3 Z endodomain
SEQ ID No. 24
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR
CD28 and CD3 Zeta endodomains
SEQ ID No. 25
SKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQE
GLYNELQKDKMAEAYSEIGMKGERRRGKGHDGLYQGLSTATKDTYDAL
HMQALPPR
CD28, OX40 and CD3 Zeta endodomains
SEQ ID No. 26
SKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR
ICOS endodomain
SEQ ID No. 27
CWLTKKKYSSSVHDPNGEYMFMRAVNTAKKSRLTDVTL
CD27 endodomain
SEQ ID No. 28
QRRKYRSNKGESPVEPAEPCHYSCPREEEGSTIPIQEDYRKPEPACSP
BTLA endodomain
SEQ ID No. 29
RRHQGKQNELSDTAGREINLVDAHLKSEQTEASTRQNSQVLLSETGIY
DNDPDLCFRMQEGSEVYSNPCLEENKPGIVYASLNHSVIGPNSRLARN
VKEAPTEYASICVRS
CD30 endodomain
SEQ ID No. 30
HRRACRKRIRQKLHLCYPVQTSQPKLELVDSRPRRSSTQLRSGASVTE
PVAEERGLMSQPLMETCHSVGAAYLESLPLQDASPAGGPSSPRDLPEP
RVSTEHTNNKIEKIYIMKADTVIVGTVKAELPEGRGLAGPAEPELEEE
LEADHTPHYPEQETEPPLGSCSDVMLSVEEEGKEDPLPTAASGK
GITR endodomain
SEQ ID No. 31
QLGLHIWQLRSQCMWPRETQLLLEVPPSTEDARSCQFPEEERGERSAE
EKGRLGDLWV
HVEM endodomain
SEQ ID No. 32
CVKRRKPRGDVVKVIVSVQRKRQEAEGEATVIEALQAPPDVTTVAVEE
TIPSFTGRSPNH

A variant sequence may have at least 80%, 85%, 90%, 95%, 98% or 99% sequence identity to SEQ ID No. 24 to 32, provided that the sequence provides an effective intracellular signalling domain.

Nucleic Acid

The present invention also provides a nucleic acid encoding a chimeric transmembrane protein of the invention.

The nucleic acid may have the structure:

AgB-spacer-TM-endo

in which

AgB1 is a nucleic acid sequence encoding the antigen-binding domain of the chimeric transmembrane protein;

spacer 1 is a nucleic acid sequence encoding the spacer of the chimeric transmembrane protein;

TM1 is a nucleic acid sequence encoding the transmembrane domain of the chimeric transmembrane protein;

endo 1 is a nucleic acid sequence encoding the endodomain of the chimeric transmembrane protein.

Alternatively the nucleic acid may have the structure:

Dim-spacer-TM-endo

in which

Dim is a nucleic acid sequence encoding the dimerisation domain of the chimeric transmembrane protein;

spacer 1 is a nucleic acid sequence encoding the spacer of the chimeric transmembrane protein;

TM1 is a nucleic acid sequence encoding the transmembrane domain of the chimeric transmembrane protein;

endo 1 is a nucleic acid sequence encoding the endodomain of the chimeric transmembrane protein.

Nucleic Acid Construct

The present invention further provides a nucleic acid construct which A nucleic acid construct encoding a chimeric cytokine receptor according to the first embodiment of the second aspect of the invention may comprise a first nucleic acid sequence encoding the first polypeptide; and a second nucleic acid sequence encoding the second polypeptide, the nucleic acid construct having the structure:

Dim1-TM1-endo1-coexpr-Dim2-TM2-endo2

in which

Dim1 is a nucleic acid sequence encoding the first dimerisation domain;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both CCRs

Dim2 is a nucleic acid sequence encoding the second dimerization domain;

TM2 is a nucleic acid sequence encoding the transmembrane domain of the second polypeptide;

endo 2 is a nucleic acid sequence encoding the endodomain of the second polypeptide.

A nucleic acid construct encoding a chimeric cytokine receptor according to the second embodiment of the second aspect of the invention may comprise a first nucleic acid sequence encoding the first polypeptide and a second nucleic acid sequence encoding the second polypeptide, the nucleic acid construct having the structure:

AgB1-spacer1-TM1-endo1-coexpr-AbB2-spacer2-TM2-endo2

in which

AgB1 is a nucleic acid sequence encoding the antigen-binding domain of the first polypeptide;

spacer 1 is a nucleic acid sequence encoding the spacer of the first polypeptide;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both polypeptides

AgB2 is a nucleic acid sequence encoding the antigen-binding domain of the second polypeptide;

spacer 2 is a nucleic acid sequence encoding the spacer of the second polypeptide;

TM2 is a nucleic acid sequence encoding the transmembrane domain of the second polypeptide;

endo 2 is a nucleic acid sequence encoding the endodomain of the second polypeptide.

A nucleic acid construct encoding a chimeric cytokine receptor according to the third embodiment of the second aspect of the invention may comprise a first nucleic acid sequence encoding the first polypeptide and a second nucleic acid sequence encoding the second polypeptide, the nucleic acid construct having the structure:

VH-spacer1-TM1-endo1-coexpr-VL-spacer2-TM2-endo2

in which

VH is a nucleic acid sequence encoding the VH domain of the first polypeptide;

spacer 1 is a nucleic acid sequence encoding the spacer of the first polypeptide;

TM1 is a nucleic acid sequence encoding the transmembrane domain of the first polypeptide;

endo 1 is a nucleic acid sequence encoding the endodomain of the first polypeptide;

coexpr is a nucleic acid sequence enabling co-expression of both polypeptides

VL is a nucleic acid sequence encoding the VL domain of the second polypeptide;

spacer 2 is a nucleic acid sequence encoding the spacer of the second polypeptide;

TM2 is a nucleic acid sequence encoding the transmembrane domain of the second polypeptide;

endo 2 is a nucleic acid sequence encoding the endodomain of the second polypeptide.

When the nucleic acid construct is expressed in a cell, such as a T-cell, it encodes a polypeptide which is cleaved at the cleavage site such that the first and second polypeptides are co-expressed at the cell surface.

Where the CCR binds a ligand, the first and second polypeptides may bind distinct epitopes on the same antigen.

The first and second polypeptides may have complementary endodomains e.g. one derived from the α or β chain of a cytokine receptor and one derived from the γ chain of the same cytokine receptor.

The present invention also provides a nucleic acid construct encoding a CCR of the invention and a CAR.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of both first and second polypeptides. It may be a sequence encoding a cleavage site, such that the nucleic acid construct produces comprises two or more CCR-forming polypeptides, or a CCR and a CAR, joined by a cleavage site(s). The cleavage site may be self-cleaving, such that when the polypeptide is produced, it is immediately cleaved into individual peptides without the need for any external cleavage activity.

The cleavage site may be any sequence which enables the first and second polypeptides, or CCR and CAR, to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

(SEQ ID No. 33)
YHADYYKQRLIHDVEMNPGP
(SEQ ID No. 34)
HYAGYFADLLIHDIETNPGP
(SEQ ID No. 35)
QCTNYALLKLAGDVESNPGP
(SEQ ID No. 36)
ATNFSLLKQAGDVEENPGP
(SEQ ID No. 37)
AARQMLLLLSGDVETNPGP
(SEQ ID No. 38)
RAEGRGSLLTCGDVEENPGP
(SEQ ID No. 39)
TRAEIEDELIRAGIESNPGP
(SEQ ID No. 40)
TRAEIEDELIRADIESNPGP
(SEQ ID No. 41)
AKFQIDKILISGDVELNPGP
(SEQ ID No. 42)
SSIIRTKMLVSGDVEENPGP
(SEQ ID No. 43)
CDAQRQKLLLSGDIEQNPGP
(SEQ ID No. 44)
YPIDFGGFLVKADSEFNPGP

The cleavage site may comprise the 2A-like sequence shown as SEQ ID No. 38 (RAEGRGSLLTCGDVEENPGP).

The present invention also provides a kit comprising one or more nucleic acid sequence(s) encoding first and second CCRs according to the first aspect of the present invention, or one or more CCR(s) according to the invention and one or more CAR(s).

SEQ ID NOS 45 and 46 give the complete amino acid sequences of a fusion between and anti-PSMA CAR and an anti-PSA CCR. Subheadings are given to label each portion of the sequence but in practice the various elements are connected giving one continuous sequence.

The nucleic acid construct of the invention may encode a fusion protein as shown in SEQ ID No. 45 or 46.

Illustrative construct with IL-2R beta chain
SEQ ID NO. 45
Signal sequence derived from human CD8a:
MSLPVTALLLPLALLLHAA
scFv aPSMA (J591 H/L)
EVQLQQSGPELKKPGTSVRISCKTSGYTFTEYTIHWVKQSHGKSLEWIGNINPNNGGTTY
NQKFEDKATLTVDKSSSTAYMELRSLTSEDSAVYYCAAGWNFDYWGQGTTL
TVSSGGGGSGGGGSGGGGSDIVMTQSHKFMSTSVGDRVSIICKASQDVGTAVDW
YQQKPGQSPKLLIYWASTRHTGVPDRFTGSGSGTDFTLTITNVQSEDLADYFCQQY
NSYPLTFGAGTMLDLKR
Linker
SDPA
Human IgG1Fc spacer (HCH2CH3pvaa):
EPKSPDKTHTCPPCPAPPVAGPSVFLFPPKPKDTLMIARTPEVTCVVVDVSHEDPEV
KFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKS
LSLSPGK
Transmembrane derived from human CD28:
FWVLVVVGGVLACYSLLVTVAFIIFWV
Endodomain derived from TCRz:
RRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQA
LPPR
2A peptide from Thosea asigna virus capsid protein:
RAEGRGSLLTCGDVEENPGP
Signal sequence derived from mouse kappa VIII:
METDTLILWVLLLLVPGSTG
scFv aPSA (5D5A5 H/L):
QVQLQQSGAELAKPGASVKMSCKTSGYSFSSYWMHWVKQRPGQGLEWIGYINPS
TGYTENNQKFKDKVTLTADKSSNTAYMQLNSLTSEDSAVYYCARSGRLYFDVWGA
GTTVTVSSGGGGSGGGGSGGGGSGGGGSDIVLTQSPPSLAVSLGQRATISCRASE
SIDLYGFTFMHWYQQKPGQPPKILIYRASNLESGIPARFSGSGSRTDFTLTINPVEAD
DVATYYCQQTHEDPYTFGGGTKLEIK
Linker:
SDPA
Human CD8aSTK spacer:
TTTPAPRPPTPAPTIASQPLSLRPEACRPAAGGAVHTRGLDFACDI
Transmembrane derived from human common gamma chain:
VVISVGSMGLIISLLCVYFWL
Endodomain derived from human common gamma chain:
ERTMPRIPTLKNLEDLVTEYHGNFSAWSGVSKGLAESLQPDYSERLCLVSEIPPKGG
ALGEGPGASPCNQHSPYWAPPCYTLKPET
2A peptide from equine rhinitis A virus polyprotein:
QCTNYALLKLAGDVESNPGP
Signal sequence derived from mouse kappa VIII:
METDTLILWVLLLLVPGSTG
scFv aPSA (5D3D11 H/L):
QVQLQQSGPELVKPGASVKISCKVSGYAISSSWMNWVKQRPGQGLEWIGRIYPGD
GDTKYNGKFKDKATLTVDKSSSTAYMQLSSLTSVDSAVYFCARDGYRYYFDYWGQ
GTSVTVSSGGGGSGGGGSGGGGSGGGGSDIVMTQTAPSVFVTPGESVSISCRSS
KSLLHSNGNTYLYWFLQRPGQSPQLLIYRMSNLASGVPDRFSGSGSGTDFTLRISR
VEAEDVGVYYCMQHLEYPVTFGAGTKVEIK
Linker:
SDPA
Human CD28STK spacer:
KIEVMYPPPYLDNEKSNGTIIHVKGKHLCPSPLFPGPSKP
Transmembrane derived from human IL-2Rβ:
IPWLGHLLVGLSGAFGFIILVYLLI
Endodomain derived from human IL-2Rβ:
NCRNTGPWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHLPDALEIEAC
QVYFTYDPYSEEDPDEGVAGAPTGSSPQPLQPLSGEDDAYCTFPSRDDLLLFSPSL
LGGPSPPSTAPGGSGAGEERMPPSLQERVPRDWDPQPLGPPTPGVPDLVDFQPP
PELVLREAGEEVPDAGPREGVSFPWSRPPGQGEFRALNARLPLNTDAYLSLQELQ
GQDPTHLV
Illustrative construct with IL-7R alpha chain
SEQ ID No. 46
Signal sequence derived from human CD8a:
MSLPVTALLLPLALLLHAA
scFv aPSMA (J591 H/L)
EVQLQQSGPELKKPGTSVRISCKTSGYTFTEYTIHWVKQSHGKSLEWIGNINPNNG
GTTYNQKFEDKATLTVDKSSSTAYMELRSLTSEDSAVYYCAAGWNFDYWGQGTTL
TVSSGGGGSGGGGSGGGGSDIVMTQSHKFMSTSVGDRVSIICKASQDVGTAVDW
YQQKPGQSPKLLIYWASTRHTGVPDRFTGSGSGTDFTLTITNVQSEDLADYFCQQY
NSYPLTFGAGTMLDLKR
Linker
SDPA
Human IgG1Fc spacer (HCH2CH3pvaa):
EPKSPDKTHTCPPCPAPPVAGPSVFLFPPKPKDTLMIARTPEVTCVVVDVSHEDPEV
KFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKS
LSLSPGK
Transmembrane derived from human CD28:
FWVLVVVGGVLACYSLLVTVAFIIFWV
Endodomain derived from TCRz:
RRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQA
LPPR
2A peptide from Thosea asigna virus capsid protein:
RAEGRGSLLTCGDVEENPGP
Signal sequence derived from mouse kappa VIII:
METDTLILWVLLLLVPGSTG
scFv aPSA (5D5A5 H/L):
QVQLQQSGAELAKPGASVKMSCKTSGYSFSSYWMHWVKQRPGQGLEWIGYINPS
TGYTENNQKFKDKVTLTADKSSNTAYMQLNSLTSEDSAVYYCARSGRLYFDVWGA
GTTVTVSSGGGGSGGGGSGGGGSGGGGSDIVLTQSPPSLAVSLGQRATISCRASE
SIDLYGFTFMHWYQQKPGQPPKILIYRASNLESGIPARFSGSGSRTDFTLTINPVEAD
DVATYYCQQTHEDPYTFGGGTKLEIK
Linker:
SDPA
Human CD8aSTK spacer:
TTTPAPRPPTPAPTIASQPLSLRPEACRPAAGGAVHTRGLDFACDI
Transmembrane derived from human common gamma chain:
VVISVGSMGLIISLLCVYFWL
Endodomain derived from human common gamma chain:
ERTMPRIPTLKNLEDLVTEYHGNFSAWSGVSKGLAESLQPDYSERLCLVSEIPPKGG
ALGEGPGASPCNQHSPYWAPPCYTLKPET
2A peptide from equine rhinitis A virus polyprotein:
QCTNYALLKLAGDVESNPGP
Signal sequence derived from mouse kappa VIII:
METDTLILWVLLLLVPGSTG
scFv aPSA (5D3D11 H/L):
QVQLQQSGPELVKPGASVKISCKVSGYAISSSWMNWVKQRPGQGLEWIGRIYPGD
GDTKYNGKFKDKATLTVDKSSSTAYMQLSSLTSVDSAVYFCARDGYRYYFDYWGQ
GTSVTVSSGGGGSGGGGSGGGGSGGGGSDIVMTQTAPSVFVTPGESVSISCRSS
KSLLHSNGNTYLYWFLQRPGQSPQLLIYRMSNLASGVPDRFSGSGSGTDFTLRISR
VEAEDVGVYYCMQHLEYPVTFGAGTKVEIK
Linker:
SDPA
Human CD28STK spacer:
KIEVMYPPPYLDNEKSNGTIIHVKGKHLCPSPLFPGPSKP
Transmembrane derived from human IL-7Rα:
PILLTISILSFFSVALLVILACVLW
Endodomain derived from human IL-7Rα:
KKRIKPIVWPSLPDHKKTLEHLCKKPRKNLNVSFNPESFLDCQIHRVDDIQARDEVEG
FLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQ
PILTSLGSNQEEAYVTMSSFYQNQ

Vector

The present invention also provides a vector, or kit of vectors, which comprises one or more nucleic acid sequence(s) encoding a one or more chimeric transmembrane protein(s) according to the first aspect of the invention and optionally one or more CAR(s). Such a vector may be used to introduce the nucleic acid sequence(s) into a host cell so that it expresses a CCR according to the second aspect of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention provides a cell which comprises one or more CCR(s) of the invention and optionally one of more CAR(s).

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CCR-expressing cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CCR-expressing cells are generated by introducing DNA or RNA coding for the or each CCR(s) by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CCR according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with one or more a nucleic acid sequence(s) encoding a CCR.

The T or NK cells may then by purified, for example, selected on the basis of expression of the antigen-binding domain of the antigen-binding polypeptide.

Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a CCR-expressing cell of the present invention for use in treating and/or preventing a disease.

The invention also relates to the use of a CCR-expressing cell of the present invention in the manufacture of a medicament for the treatment and/or prevention of a disease.

The disease to be treated and/or prevented by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

Where the ligand recognised by the CCR is PSA, the cancer may be prostate cancer.

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

The cells and pharmaceutical compositions of present invention may be for use in any of the methods described above.

The invention will now be further described by way of Examples, which are meant to serve to assist one of ordinary skill in the art in carrying out the invention and are not intended in any way to limit the scope of the invention.

### EXAMPLES

### Example 1—In Vitro Testing

T-cells are transduced with either a PSMA-specific CAR, or transduced with a construct which co-expresses a PSMA-specific CAR with a PSA-specific CCR. T-cells are co-cultured with PSMA expressing target cells which secrete or do not secrete PSA. This co-culture is conducted in the presence or absence of exogenous IL2. This co-culture is conducted at different effector to target ratios. This co-culture is repeated serially with T-cells challenged with repeated target cells. Proliferation of T-cells and killing of target cells is determined. In this way, the contribution to proliferation and survival of T-cells the CCR makes can be measured. Further, the ability contribution to repeated re-challenge the ability of serial

### Example 2—In Vivo Testing

NSG mice are engrafted with a human prostate cancer cell line which expresses PSMA and secretes PSA and which expresses firefly Luciferase. T-cells are transduced with either a PSMA-specific CAR, or transduced with a construct which co-expresses the PSMA-specific CAR with a PSA-specific CCR. T-cells are administered to the mice. The tumour burden can be serially measured using bioluminescent imaging and the response to CAR T-cells evaluated. Mice within each cohort can be sacrificed at different time-points and tumour burden directly measured by macroscopic measurements and by immunohistochemistry. Further, engraftment/expansion of T-cells at the tumour bed or within lymphoid tissues such as lymph nodes, spleen and bone-marrow measured by flow cytometry of said tissues.

### Example 3—Creation of and Testing a Constitutively Active Cytokine-Signalling Molecule

A constitutively active cytokine-signalling chimeric transmembrane protein was produced by linking cytokine receptor endodomains to a “Fab” type exodomain (FIG. 5). This structure uses the natural dimerization components of antibodies, namely the dimerization domain from the heavy and light chain constant regions. The chimeric transmembrane protein has two chains; a first polypeptide which comprises the antibody light κ chain and the IL2 receptor common γ chain as endodomain; and a second polypeptide which comprises the antibody heavy chain CH1 and an endodomain which comprises either: the IL2 receptor β chain (giving a constitutively active IL2-signalling molecule); or the IL7 receptor (giving a constitutively active IL7-signalling molecule). The constitutively active cytokine-signalling chimeric transmembrane proteins tested in this study included the scFv heavy and light chain variable regions. These domains are not needed for dimerization to occur. The signal is independent of antigen binding and the structure could equally be “headless” (as shown in FIG. 5) or comprise another entity such as a protein tag.

Nucleic acid sequences encoding these two polypeptides were cloned in frame separated by a 2A-peptide encoding sequence.

CTLL-2 (ATCC® TIB-214™) are murine cytotoxic T lymphocyte cells which are dependent upon IL-2 for growth. In the absence of IL-2 the cells undergo apoptosis.

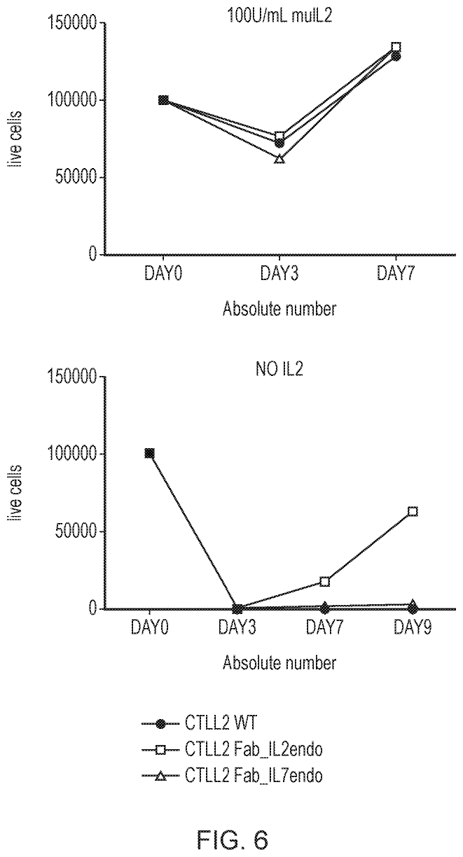

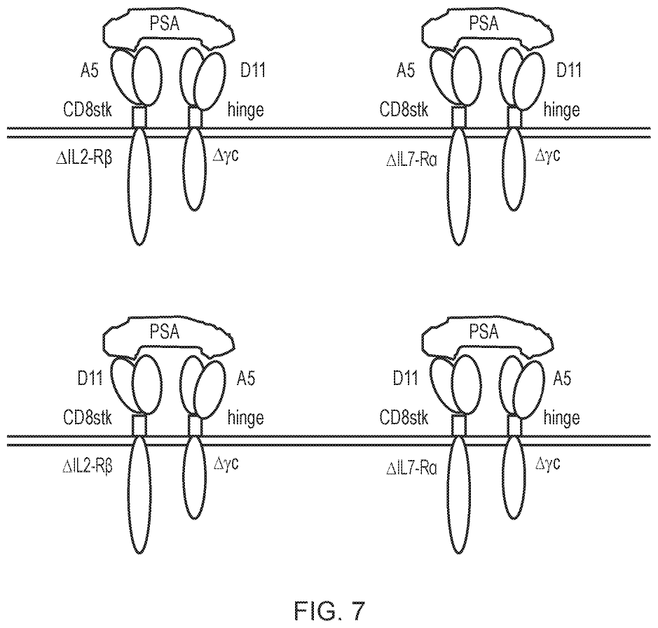

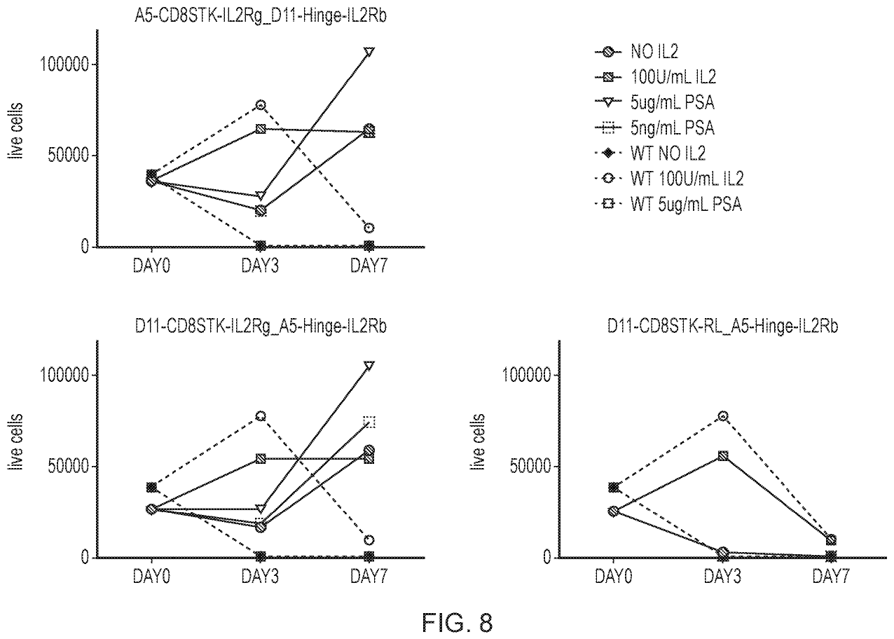

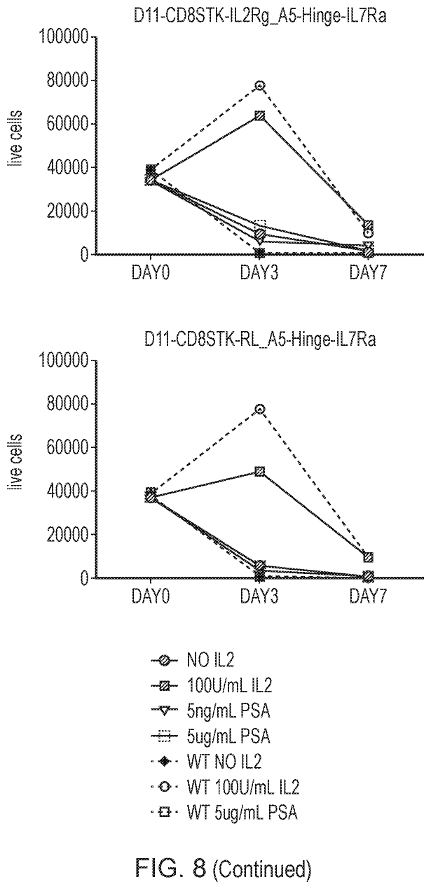

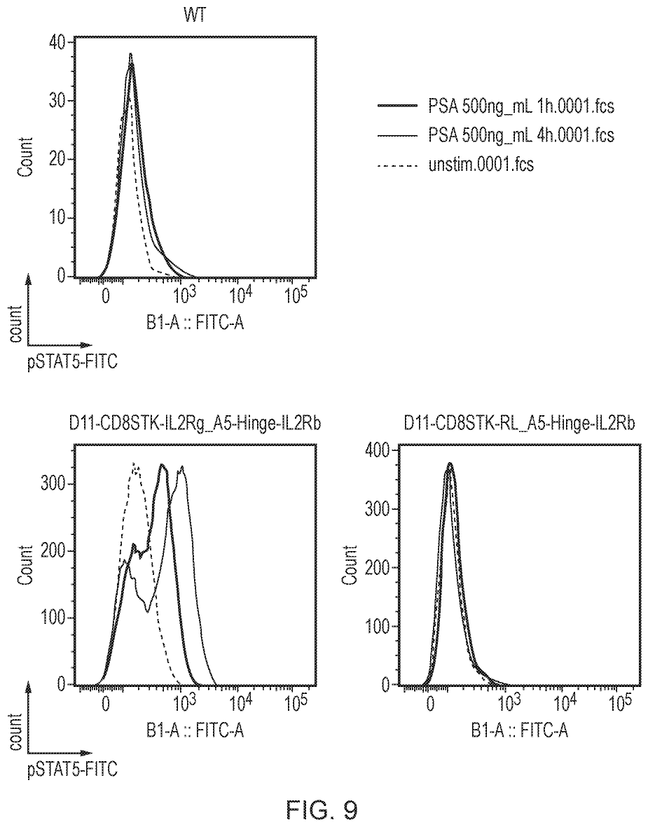

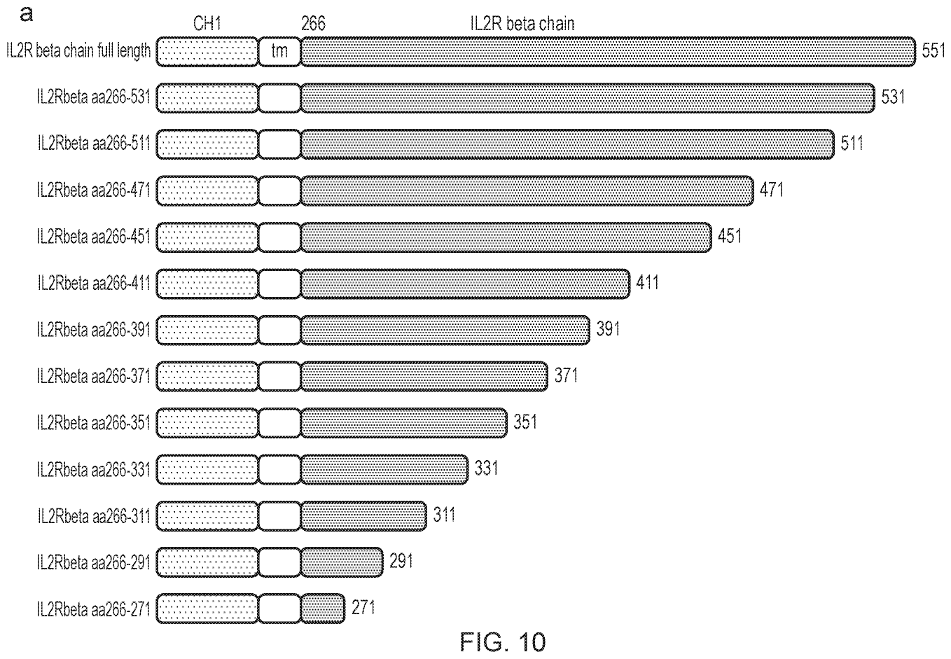

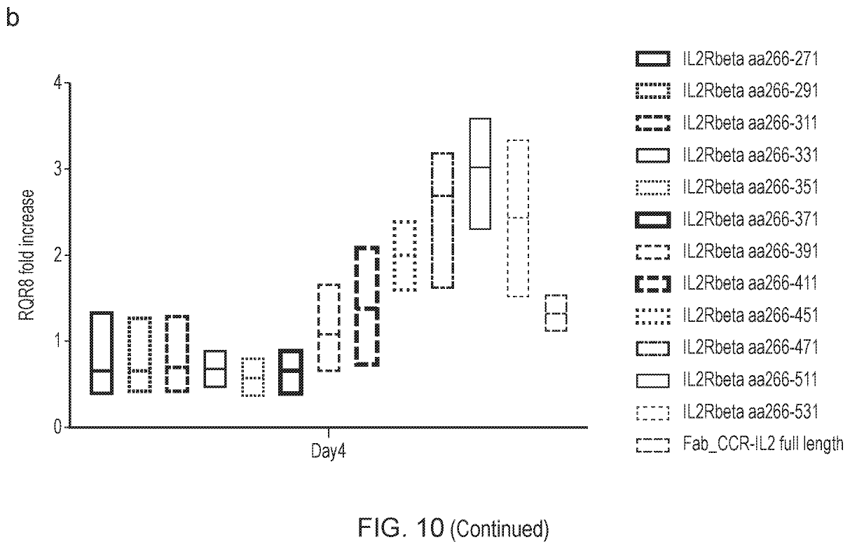

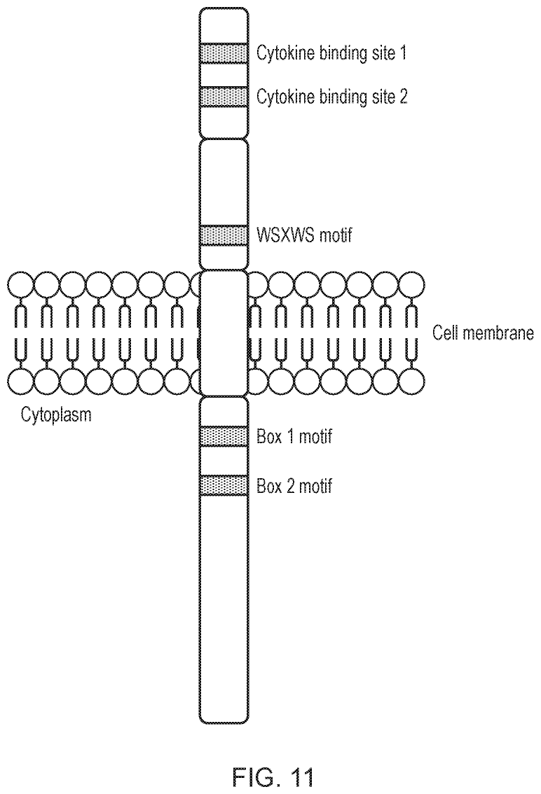

CTLL-2 cells were transduced with a vector expressing the chimeric protein comprising an IL2-receptor endodomain (Fab_IL2endo) or a vector expressing the chimeric protein comprising an IL7 receptor endodomain (Fab_IL7endo) or left untransduced (WT). As a positive control, cells of all three types were co-cultured with 100 U/ml murine IL2. Cell proliferation was assessed after 3 and 7 days of culture and the results are shown in FIG. 6.

Untransduced CTLL2 cells, together with CTLL2 cells transduced with either construct (Fab_IL2endo or Fab_IL7endo) proliferated in the presence of 100 U/mL murine IL2 (FIG. 6, left-hand panel). However, in the absence of exogenously added IL2, only cells transduced with the construct having an IL2R endodomain (Fab_IL2endo) survived and proliferated. This shows that the chimeric transmembrane receptor provides the CTLL2 cells with the necessary IL2 signal.

### Example 4—Generation and Testing of a Chimeric Cytokine Receptor Against PSA

A panel of chimeric cytokine receptors targeting PSA was developed using scFvs derived from two antibodies which bind to different PSA epitopes: 5D5A5 and 5D3D11. The crystal structure of PSA has been obtained in a sandwich complex with these two (Stura et al (2011) as above).

Schematic diagrams illustrating some of the panel of CCRs is illustrated in FIG. 7.

The panel included the following constructs:

A5-CD8stk-IL2Rg_D11-Hinge-IL2Rb: A CCR with an IL-2R endodomain having A5 on the chain with common γ chain and D11 on the chain with the IL2R β chain;

D11-CD8stk-IL2Rg_A5-Hinge-IL2Rb: A CCR with an IL-2R endodomain having D11 on the chain with common γ chain and A5 on the chain with IL2R β chain;

D11-CD8stk-RL_A5-Hinge-IL2Rb: A negative control construct which is equivalent D11-CD8stk-IL2Rg_A5-Hinge-IL2Rb, but in which the IL2Rγ chain is replaced by a rigid linker;

D11-CD8stk-IL2Rg_A5-Hinge-IL7Ra: A CCR with an IL-7R endodomain having D11 on the chain with common γ chain and A5 on the chain with IL7R α chain; and

D11-CD8stk-RL_A5-Hinge-IL7Ra: A negative control construct which is equivalent D11-CD8stk-IL2Rg_A5-Hinge-IL7Ra, but in which the IL2Rγ chain is replaced by a rigid linker;

CTLL2 cells were transduced with vectors expressing these constructs. Cells were cultured in the presence or absence of IL2 (the presence of IL2 acting as a positive control) and the presence or absence of 5 ng/mL or 5 μg/mL PSA. CTLL2 cell proliferation was assessed after 3 and 7 days and the results are shown in FIG. 8.

CTLL2 cells expressing a CCR with an IL7 endodomain did not support CTLL2 cell survival and proliferation (FIG. 8, last two panels). The presence of murine IL-2 in these cells supported CTLL2 cell growth and proliferation at day 3, but by day 7 the majority of cells had undergone apoptosis.

The anti-PSA chimeric cytokine receptors with an IL2R endodomain supported CTLL2 cell proliferation in the absence of IL2 and the presence of PSA at both 5 ng/ml and 5 μg/ml (FIG. 8, first panel), with 5 μg/ml giving greater survival and proliferation, particularly at day 7.

Both the anti-PSA chimeric cytokine receptors with an IL2R endodomain, i.e. A5-CD8stk-IL2Rg_D11-Hinge-IL2Rb and D11-CD8stk-IL2Rg_A5-Hinge-IL2Rb, indicating that the relative positioning of the two PSA-binding domains: 5D5A5 and 5D3D11, is not important for function.

Substitution of the common γ chain with a rigid linker abolished the capacity of the CCR to support CTLL2 cell survival and proliferation (FIG. 8, third panel).

As another read-out for IL2 signalling, the phosphorylation of Y694 of STAT5 was investigated using phosphoflow.

CTLL2 cells were either untransduced (WT); transduced with a PSA CCR constructs having an IL2R endodomain (D11-CD8STK-IL2Rg_A5-Hinge-IL2Rb); or transduced with an equivalent negative control construct in which the IL2Rγ chain is replaced with a rigid linker (D11-CD8STK-RL_A5-Hinge-IL2Rb). The cells were incubated overnight in the absence of exogenously added IL-2. The following day, the cells were incubated with either Pervanadate at 500 μM (a positive control which inhibits phosphatase and will lead to STAT5 phosphorylation) or 500 ng/mL PSA for 1 or 4 hours. After incubation the cells were fixed, permeabilised and analysed by flow cytometry.

The results are shown in FIG. 9. In the cells expressing the PSA CCR, the presence of PSA lead to increasing STAT5 phosphorylation with time (FIG. 9, central panel). No such increase in phosphorylation was seen with untransduced CTLL2 cells, or with CTLL2 cells transduced with an equivalent construct in which the IL2Rγ chain is replaced with a rigid linker (FIG. 9, right hand panel).

These results are consistent with the CTLL2 survival/proliferation data shown in FIG. 8 and demonstrate that a chimeric cytokine receptor against a soluble ligand (here, PSA) can be used to trigger cytokine signalling in a T-cell.

### Example 5—Generation and Testing of a Chimeric Cytokine Receptor with a Truncated IL-2 Receptor β-Chain Endodomain

Constructs were created having the general structure:

RQR8-2A-CL-SP1-TM1-IL2Rγ-2Aw-SP2-CH-TM2-IL2Rβ

in which:

RQR8 is a marker gene described in WO2013/153391

2A and 2Aw are self-cleaving peptides: the sequence encoding 2Aw is codon wobbled to prevent homologous recombination

CL is Light kappa chain

SP1 and SP2 are spacers

TM1 and TM2 are transmembrane domains

IL2Rγ is the endodomain from IL2R common gamma chain

CH is heavy chain constant region

IL2Rβ is a truncated or full-length IL-2 receptor β-chain endodomain

Constructs were generated with the series of truncated IL-2 receptor β-chain endodomain shown in FIG. 10a and the key for FIG. 10b.

T cells were transduced with vectors expressing each construct and cultured for 4 days in absence of exogenous cytokines (starvation assay). The absolute number of viable, transduced cells was assessed by flow cytometry. The results are shown in FIG. 10b. Truncation of the IL-2 receptor β-chain endodomain by 20 or 40 amino acids (i.e. from amino acids 266-551 to 266-531 and 266-511 respectively) increased proliferation, with the highest level of proliferation observed for the IL2Rbeta aa266-511. Further truncation of the IL-2 receptor β-chain endodomain results in a step-wise reduction in proliferation from aa266-471>aa266-451>aa 266-411>aa266-391>aa266-371, at which point it plateaued with further deletions having no significant effect of the level of proliferation. It is therefore possible to reduce the activity of chimeric cytokine receptors by truncation of one or both cytokine receptor endodomains. It is also possible to “tailor” CCRs to have a desired level of cytokine production (in this case IL-2) by selecting an endodomain truncation which gives the desired level of activity.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-2. (canceled)

3. A chimeric cytokine receptor, which comprises two polypeptides:
(i) a first polypeptide which comprises:
(a) a first dimerisation domain; and
(b) a first chain of the cytokine receptor endodomain; and
(ii) a second polypeptide which comprises:
(a) a second dimerization domain, which dimerises with the first dimerization domain; and
(b) a second chain of the cytokine-receptor endodomain
wherein the first and/or second chain of the cytokine-receptor endodomain is/are truncated.

4. A chimeric cytokine receptor according to claim 3, wherein the first and second dimerization domains dimerise spontaneously.

5. A chimeric cytokine receptor according to claim 3, where the first and second dimerization domains dimerise in the presence of a chemical inducer of dimerization (CID) or the presence of a protein.

6. A chimeric cytokine receptor according to claim 4 which comprises two polypeptides:
(i) a first polypeptide which comprises:
(a) a heavy chain constant domain (CH)
(b) a first chain of the cytokine receptor endodomain; and
(ii) a second polypeptide which comprises:
(a) a light chain constant domain (CL)
(b) a second chain of the cytokine-receptor endodomain.

7. A chimeric cytokine receptor comprising:
an exodomain which binds to a ligand; and
a cytokine receptor endodomain comprising a first chain and a second chain
wherein the first and/or second chain of the cytokine-receptor endodomain is/are truncated.

8. A chimeric cytokine receptor according to claim 7, which comprises two polypeptides:
(i) a first polypeptide which comprises:
(a) a first antigen-binding domain which binds a first epitope of the ligand
(b) a first chain of the cytokine receptor endodomain; and
(ii) a second polypeptide which comprises:
(a) a second antigen-binding domain which binds a second epitope of the ligand
(b) a second chain of the cytokine-receptor endodomain.

9-13. (canceled)

14. A chimeric cytokine receptor according to claim 3 wherein the first and second chains of the cytokine receptor endodomain are selected from type I cytokine receptor endodomain α-, β-, and γ-chains.

15. A chimeric cytokine receptor according to claim 14, wherein the first and second chains of the cytokine receptor endodomain are selected from:
(i) IL-2 receptor β-chain endodomain
(ii) IL-7 receptor α-chain endodomain; or
(iii) IL-15 receptor α-chain endodomain; and/or
(iv) common γ-chain receptor endodomain.

16. A chimeric cytokine receptor according to claim 3, which comprises a truncated IL-2 receptor β-chain endodomain.

17. A cell which comprises a chimeric cytokine receptor according to claim 3.

18. A cell according to claim 17, which also comprises a chimeric antigen receptor.

19. (canceled)

20. A nucleic acid construct encoding a chimeric cytokine receptor according to claim 3 which comprises a first nucleic acid sequence encoding the first polypeptide; and a second nucleic acid sequence encoding the second polypeptide, the nucleic acid construct having the structure:
Dim1-TM1-endo1-coexpr-Dim2-TM2-endo2
in which
Dim1 is a nucleic acid sequence encoding the first dimerisation domain;
TM1 is a nucleic acid sequence encoding the transmembrane domain of the first polypeptide;
endo 1 is a nucleic acid sequence encoding the endodomain of the first polypeptide;
coexpr is a nucleic acid sequence enabling co-expression of both CCRs
Dim2 is a nucleic acid sequence encoding the second dimerization domain;
TM2 is a nucleic acid sequence encoding the transmembrane domain of the second polypeptide;
endo 2 is a nucleic acid sequence encoding the endodomain of the second polypeptide.

21. A nucleic acid construct encoding a chimeric cytokine receptor according to claim 7, which comprises a first nucleic acid sequence encoding the first polypeptide and a second nucleic acid sequence encoding the second polypeptide, the nucleic acid construct having the structure:
AgB1-spacer1-TM1-endo1-coexpr-AbB2-spacer2-TM2-endo2
in which
AgB1 is a nucleic acid sequence encoding the antigen-binding domain of the first polypeptide;
spacer 1 is a nucleic acid sequence encoding the spacer of the first polypeptide;
TM1 is a nucleic acid sequence encoding the transmembrane domain of the first polypeptide;
endo 1 is a nucleic acid sequence encoding the endodomain of the first polypeptide;
coexpr is a nucleic acid sequence enabling co-expression of both polypeptides
AgB2 is a nucleic acid sequence encoding the antigen-binding domain of the second polypeptide;
spacer 2 is a nucleic acid sequence encoding the spacer of the second polypeptide;
TM2 is a nucleic acid sequence encoding the transmembrane domain of the second polypeptide;
endo 2 is a nucleic acid sequence encoding the endodomain of the second polypeptide.

22-25. (canceled)

26. A vector comprising a nucleic acid construct according to claim 20.

27-28. (canceled)

29. A method for making a cell according to claim 17, which comprises the step of introducing into a cell:
(i) a first nucleic acid sequence encoding a first polypeptide which comprises:
(a) a first dimerisation domain; and
(b) a first chain of the cytokine receptor endodomain; and
(ii) a second nucleic acid sequence encoding a second polypeptide which comprises:
(a) a second dimerization domain, which dimerises with the first dimerization domain; and
(b) a second chain of the cytokine-receptor endodomain
wherein the first and/or second chain of the cytokine-receptor endodomain is/are truncated.

30. (canceled)

31. A pharmaceutical composition comprising a plurality of cells according to claim 17.

32. A method for treating and/or preventing a disease, which comprises the step of administering a pharmaceutical composition according to claim 31 to a subject.

33-37. (canceled)

38. A cell which comprises a chimeric cytokine receptor according to claim 7.

39. A vector comprising a nucleic acid construct according to claim 21.


SEQUENCE LISTING (71 sequences)

SEQ ID NO: 1 - protein, 86 aa, Artificial Sequence
  endodomain derived from human IL-2 receptor common gamma chain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, Artificial Sequence
  endodomain derived from human IL-2 receptor beta chain (IL-2Rbeta)
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, Artificial Sequence
  endodomain derived from human IL-7Ralpha
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 5 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 6 - protein, 20 aa, Artificial Sequence
  spacer sequence, human IgG1 hinge
AEPKSPDKTHTCPPCPKDPK

SEQ ID NO: 7 - protein, 21 aa, Artificial Sequence
  transmembrane derived from human common gamma chain
VVISVGSMGLIISLLCVYFWL

SEQ ID NO: 8 - protein, 25 aa, Artificial Sequence
  transmembrane derived from human IL-2Rbeta
IPWLGHLLVGLSGAFGFIILVYLLI

SEQ ID NO: 9 - protein, 25 aa, Artificial Sequence
  transmembrane derived from human IL-7Ralpha
PILLTISILSFFSVALLVILACVLW

SEQ ID NO: 10 - protein, 22 aa, Artificial Sequence
  transmembrane derived from human IL-15Ralpha
AISTSTVLLCGLSAVSLLACYL

SEQ ID NO: 11 - protein, 118 aa, Artificial Sequence
  PSA-binding mAb 5D3D11 heavy chain variable domain (VH)
QVQLQQSGPELVKPGASVKISCKVSGYAISSSWMNWVKQRPGQGLEWIGRIYPGDGDTKYNGKFKDKATLTVDKSSSTAYMQLSSLTSVDSAVYFCARDGYRYYFDYWGQGTSVTVSS

SEQ ID NO: 12 - protein, 112 aa, Artificial Sequence
  PSA-binding mAb 5D3D11 light chain variable domain (VL)
DIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIK

SEQ ID NO: 13 - protein, 117 aa, Artificial Sequence
  PSA-binding mAb 5D5A5 VH
QVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSS

SEQ ID NO: 14 - protein, 111 aa, Artificial Sequence
  PSA-binding mAb 5D5A5 VL
DIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIK

SEQ ID NO: 15 - protein, 248 aa, Artificial Sequence
  single-chain variable fragment (scFv) based on 5D5A5
QVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSSGGGGSGGGGSGGGGSGGGGSDIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIK

SEQ ID NO: 16 - protein, 250 aa, Artificial Sequence
  scFv based on 5D3D11
QVQLQQSGPELVKPGASVKISCKVSGYAISSSWMNWVKQRPGQGLEWIGRIYPGDGDTKYNGKFKDKATLTVDKSSSTAYMQLSSLTSVDSAVYFCARDGYRYYFDYWGQGTSVTVSSGGGGSGGGGSGGGGSGGGGSDIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIK

SEQ ID NO: 17 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 18 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 19 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 20 - protein, 238 aa, Artificial Sequence
  J591 single-chain variable fragment (scFv)
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKR

SEQ ID NO: 21 - protein, 21 aa, Artificial Sequence
  signal peptide derived from TCR beta chain
MGTSLLCWMALCLLGADHADG

SEQ ID NO: 22 - protein, 21 aa, Artificial Sequence
  signal peptide derived from IgG1
MSLPVTALLLPLALLLHAARP

SEQ ID NO: 23 - protein, 20 aa, Artificial Sequence
  signal peptide derived from CD8a
MAVPTQVLGLLLLWLTDARC

SEQ ID NO: 24 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 152 aa, Artificial Sequence
  CD28 and CD3 Zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 188 aa, Artificial Sequence
  CD28, OX40 and CD3 Zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 38 aa, Artificial Sequence
  ICOS endodomain
CWLTKKKYSSSVHDPNGEYMFMRAVNTAKKSRLTDVTL

SEQ ID NO: 28 - protein, 48 aa, Artificial Sequence
  CD27 endodomain
QRRKYRSNKGESPVEPAEPCHYSCPREEEGSTIPIQEDYRKPEPACSP

SEQ ID NO: 29 - protein, 111 aa, Artificial Sequence
  BTLA endodomain
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 30 - protein, 188 aa, Artificial Sequence
  CD30 endodomain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 31 - protein, 58 aa, Artificial Sequence
  GITR endodomain
QLGLHIWQLRSQCMWPRETQLLLEVPPSTEDARSCQFPEEERGERSAEEKGRLGDLWV

SEQ ID NO: 32 - protein, 60 aa, Artificial Sequence
  HVEM endodomain
CVKRRKPRGDVVKVIVSVQRKRQEAEGEATVIEALQAPPDVTTVAVEETIPSFTGRSPNH

SEQ ID NO: 33 - protein, 20 aa, Artificial Sequence
  cleavage site, 2A-like sequence
YHADYYKQRLIHDVEMNPGP

SEQ ID NO: 34 - protein, 20 aa, Artificial Sequence
  cleavage site, 2A-like sequence
HYAGYFADLLIHDIETNPGP

SEQ ID NO: 35 - protein, 20 aa, Artificial Sequence
  cleavage site, 2A-like sequence
QCTNYALLKLAGDVESNPGP

SEQ ID NO: 36 - protein, 19 aa, Artificial Sequence
  cleavage site, 2A-like sequence
ATNFSLLKQAGDVEENPGP

SEQ ID NO: 37 - protein, 19 aa, Artificial Sequence
  cleavage site, 2A-like sequence
AARQMLLLLSGDVETNPGP

SEQ ID NO: 38 - protein, 20 aa, Artificial Sequence
  cleavage site, 2A-like sequence
RAEGRGSLLTCGDVEENPGP

SEQ ID NO: 39 - protein, 20 aa, Artificial Sequence
  cleavage site, 2A-like sequence
TRAEIEDELIRAGIESNPGP

SEQ ID NO: 40 - protein, 20 aa, Artificial Sequence
  cleavage site, 2A-like sequence
TRAEIEDELIRADIESNPGP

SEQ ID NO: 41 - protein, 20 aa, Artificial Sequence
  cleavage site, 2A-like sequence
AKFQIDKILISGDVELNPGP

SEQ ID NO: 42 - protein, 20 aa, Artificial Sequence
  cleavage site, 2A-like sequence
SSIIRTKMLVSGDVEENPGP

SEQ ID NO: 43 - protein, 20 aa, Artificial Sequence
  cleavage site, 2A-like sequence
CDAQRQKLLLSGDIEQNPGP

SEQ ID NO: 44 - protein, 20 aa, Artificial Sequence
  cleavage site, 2A-like sequence
YPIDFGGFLVKADSEFNPGP

SEQ ID NO: 45 - protein, 1722 aa, Artificial Sequence
  illustrative construct with IL-2R beta chain
MSLPVTALLLPLALLLHAAEVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMETDTLILWVLLLLVPGSTGQVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSSGGGGSGGGGSGGGGSGGGGSDIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIKSDPATTTPAPRPPTPAPTIASQPLSLRPEACRPAAGGAVHTRGLDFACDIVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETQCTNYALLKLAGDVESNPGPMETDTLILWVLLLLVPGSTGQVQLQQSGPELVKPGASVKISCKVSGYAISSSWMNWVKQRPGQGLEWIGRIYPGDGDTKYNGKFKDKATLTVDKSSSTAYMQLSSLTSVDSAVYFCARDGYRYYFDYWGQGTSVTVSSGGGGSGGGGSGGGGSGGGGSDIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIKSDPAKIEVMYPPPYLDNEKSNGTIIHVKGKHLCPSPLFPGPSKPIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 46 - protein, 1631 aa, Artificial Sequence
  illustrative construct with IL-7R alpha chain
MSLPVTALLLPLALLLHAAEVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSGGGGSGGGGSGGGGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMETDTLILWVLLLLVPGSTGQVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSSGGGGSGGGGSGGGGSGGGGSDIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIKSDPATTTPAPRPPTPAPTIASQPLSLRPEACRPAAGGAVHTRGLDFACDIVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETQCTNYALLKLAGDVESNPGPMETDTLILWVLLLLVPGSTGQVQLQQSGPELVKPGASVKISCKVSGYAISSSWMNWVKQRPGQGLEWIGRIYPGDGDTKYNGKFKDKATLTVDKSSSTAYMQLSSLTSVDSAVYFCARDGYRYYFDYWGQGTSVTVSSGGGGSGGGGSGGGGSGGGGSDIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIKSDPAKIEVMYPPPYLDNEKSNGTIIHVKGKHLCPSPLFPGPSKPPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 47 - protein, 8 aa, Artificial Sequence
  endodomain derived from human IL-2Rbeta Box 1 motif
KCNTPDPS

SEQ ID NO: 48 - protein, 11 aa, Artificial Sequence
  endodomain derived from human IL-2Rbeta Box 2 motif
SPLEVLERDKV

SEQ ID NO: 49 - protein, 68 aa, Artificial Sequence
  truncated IL-2Rbeta aa266-331
NCRNTGPWLKKVLKCNTPDPSKFFSQLSSEHGGDVQKWLSSPFPSSSFSPGGLAPEISPLEVLERDKV

SEQ ID NO: 50 - protein, 86 aa, Artificial Sequence
  truncated IL-2Rbeta aa266-351
NCRNTGPWLKKVLKCNTPDPSKFFSQLSSEHGGDVQKWLSSPFPSSSFSPGGLAPEISPLEVLERDKVTQLLLQQDKVPEPASLSS

SEQ ID NO: 51 - protein, 105 aa, Artificial Sequence
  truncated IL-2Rbeta aa266-371
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLP

SEQ ID NO: 52 - protein, 126 aa, Artificial Sequence
  truncated IL-2Rbeta aa266-391
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEED

SEQ ID NO: 53 - protein, 146 aa, Artificial Sequence
  truncated IL-2Rbeta aa266-411
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPL

SEQ ID NO: 54 - protein, 166 aa, Artificial Sequence
  truncated IL-2Rbeta aa266-431
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFS

SEQ ID NO: 55 - protein, 186 aa, Artificial Sequence
  truncated IL-2Rbeta aa266-451
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAG

SEQ ID NO: 56 - protein, 206 aa, Artificial Sequence
  truncated IL-2Rbeta aa266-471
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQP

SEQ ID NO: 57 - protein, 227 aa, Artificial Sequence
  truncated IL-2Rbeta aa266-491
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELV

SEQ ID NO: 58 - protein, 246 aa, Artificial Sequence
  truncated IL-2Rbeta aa266-511
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSF

SEQ ID NO: 59 - protein, 266 aa, Artificial Sequence
  truncated IL-2Rbeta aa266-531
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPL

SEQ ID NO: 60 - protein, 38 aa, Artificial Sequence
  endodomain derived from human IL-15Ralpha
SRQTPPLASVEMEAMEALPVTWGTSSRDEDLENCSHHL

SEQ ID NO: 61 - protein, 5 aa, Artificial Sequence
  cytokine receptor motif
  misc_feature (3)..(3): Xaa can be any naturally occurring amino acid
WSXWS

SEQ ID NO: 62 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 63 - protein, 7 aa, Artificial Sequence
  consensus Tobacco Etch Virus (TEV) cleavage site
ENLYFQS

SEQ ID NO: 64 - protein, 19 aa, Artificial Sequence
  signal sequence derived from human CD8a
MSLPVTALLLPLALLLHAA

SEQ ID NO: 65 - protein, 4 aa, Artificial Sequence
  linker
SDPA

SEQ ID NO: 66 - protein, 231 aa, Artificial Sequence
  human IgG1Fc spacer (HCH2CH3pvaa)
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 27 aa, Artificial Sequence
  transmembrane derived from human CD28
FWVLVVVGGVLACYSLLVTVAFIIFWV

SEQ ID NO: 68 - protein, 113 aa, Artificial Sequence
  endodomain derived from TCRz
RRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 20 aa, Artificial Sequence
  signal sequence derived from mouse kappa VIII
METDTLILWVLLLLVPGSTG

SEQ ID NO: 70 - protein, 250 aa, Artificial Sequence; identical to SEQ ID NO: 16
  scFv aPSA (5D3D11 H/L)

SEQ ID NO: 71 - protein, 40 aa, Artificial Sequence
  human CD28STK spacer
KIEVMYPPPYLDNEKSNGTIIHVKGKHLCPSPLFPGPSKP